(19) United States
(12) Patent Application Publication
(10) Pub. No.: US 2004/0033967 A1
(43) Pub. Date: Feb. 19, 2004

(54) Alkylated hexitol nucleoside analogues and oligomers thereof

(76) Inventors: Arthur Van Aerschot, Heist-op-den-Berg (DE); Piet Herdewijn, Wezemaal (BE)

Correspondence Address: NATH & ASSOCIATES, 1030 15th STREET, 6TH FLOOR, WASHINGTON, DC, 20005, US

(21) Appl. No.: 10/362,660

(22) PCT Filed: Aug. 28, 2001
(86) PCT No.: PCT/BE01/00143

(30) Foreign Application Priority Data
Aug. 30, 2000 (GB) 0021221.7

(51) Int. Cl. A61K 31/7052; C07H 15/00; C07H 5/04

(52) U.S. Cl. 514/25; 536/18.5; 536/18.7

(57) ABSTRACT

The present invention is directed to nucleoside analogues with as substitute for the sugar part a 1,5-anhydrohexitol moiety, doexygenated and substituted with a nucleobase at the 2-position, of which the hexitorl ring is further substituted with at least one alkoxy substituent at the 3-position or at the 1-position, and to oligonucleotides wherein at least some of the nucleotides are part of the afore mentioned hexitol nucleoside analogues and exhibit sequence-specific hydridization to complementary sequences of nucleic acids, and maintaining or improving the hybridisation strength. The invention further relates to nucleoside analogues with a 1,5-anhydrohexitol moiety as the sugar part, deoxygenated and substituted with a nucleobase at the 2-position, of which the hexitol ring is substituted with a methoxy substituent at the 1-position, having at the same time either a hydroxy or an alkoxy group at the 3-position, or having a 3-deoxygenated position. The inclusion of one or more of the afore mentioned hexitol nucleoside analogues in oligonucleotides provides, inter alia, either for improved binding or for maintained binding of these oligonucleotides to a complementary strand. This invention further relates to the chemical synthesis of these oligomers which are useful diagnostics, therapeutics and as research agents.

### FIELD OF THE INVENTION

This invention relates to the chemical synthesis of particular oligomers which are useful for diagnostics, therapeutics and as research agents.

### TECHNICAL BACKGROUND

Control of translation processes is a continuously growing research area and the use of antisense oligonucleotides reflects one of the possibilities enabling such control. This relies mostly on degradation of the mRNA target through assistance of RNase H, becoming activated upon recognition of the mixed DNA-RNA duplex. Oligonucleotides which do not activate RNase H after hybridizing with complementary RNA have to rely on a strong association with their nucleic acids target to obtain an antisense effect. If oligomers can be obtained which are able to induce strand displacement in double stranded RNA structures, targeting of RNA becomes independent of the secondary and tertiary structure of the mRNA and the number of possible RNA targets will increase considerably.

Hybridisation is the sequence specific base pair hydrogen bonding of bases of the oligonucleotide to bases of target RNA or DNA Such base pairs are said to be complementary to one another. In determining the extent of hybridization of an oligonucleotide to a complementary nucleic acid, the relative ability of an oligonucleotide to bind to the complementary nucleic acid may be compared by determining the melting temperature of a particular hybridisation complex. The melting temperature (Tₘ), a characteristic physical property of double helices, denotes the temperature in degrees centigrade, at which 50% helical (hybridized) versus coil (unhybridized) forms are present. Tₘ is measured by using the UV spectrum to determine the formation and breakdown (melting) of the hybridisation complex. Base stacking which occurs during hybridisation, is accompanied by a reduction in UV absorption (hypochromicity). Consequently, a reduction in UV absorption indicates a higher Tₘ. The higher the Tₘ, the greater the strength of the bonds between the strands.

One way to obtain strong hybridisation and consequently a high Tₘ, is to synthesize constrained carbohydrate modified oligonucleotides exemplified by hexitol nucleic acids ([1] Van Aerschot et al., Angew. Chem. Int. Ed. 1995, 34, 1338; [2] Hendrix et al., Chemistry: European J., 1997, 3, 110; [3] Hendrix, et al., Chemistry: European J., 1997, 3, 1513; [4] Boudou et al., Nucleic Acids Res., 1999, 27, 1450) or by 2′-O-(2-methoxy)ethyl moieties containing oligonucleotides ([5] Martin, P. Helv. Chim. Acta 1995, 78, 486), or by bicyclic oligonucleotides ([6] Tarköy et al., Helv. Chim. Acta 1993, 76, 481) with the compounds of the Wengel group ([7] Singh et al., J. Chem. Commun. 1998, 455) showing the strongest affinity for RNA. The strong hybridization characteristics with complementary RNA are generally attributed ([8] Herdewijn, P. Liebigs Ann. 1996, 1337) to the formation of a preorganized conformation which fits the A-form of dsRNA, good stacking interactions between the bases which interact in a Watson-Crick type geometry with their complement and efficient hydration of the double stranded helix.

Hexitol nucleic acids (HNA) are composed of phosphorylated 2,3-dideoxy-D-arabino-hexitol units with a nucleobase situated in the 2-[S]-position. They hybridize sequence-selectively with RNA in an antiparallel way. The observed increase in Tm per modification of a HNA:RNA duplex versus duplexes of natural nucleic acids is sequence- and length-dependent and varies from +0.9° C./modification to +5.8° C./modification (as described in references [2] and [3]). HNA is an efficient steric blocking agent as observed during investigations of HNA in cell-free translation experiments affording IC₅₀ values of 50 nM as inhibitors of Ha-ras mRNA translation ([9] Vandermeeren et al., Biochem Pharmacol., 2000, 59, 655). Valuable results in cellular systems recently likewise have been reported as there are the inhibition of Ha-ras and ICAM-1 (see reference [9]), and antimalarial activity ([10] Flores et al., Parasitol. Res. Series, 1999, 85, 864).

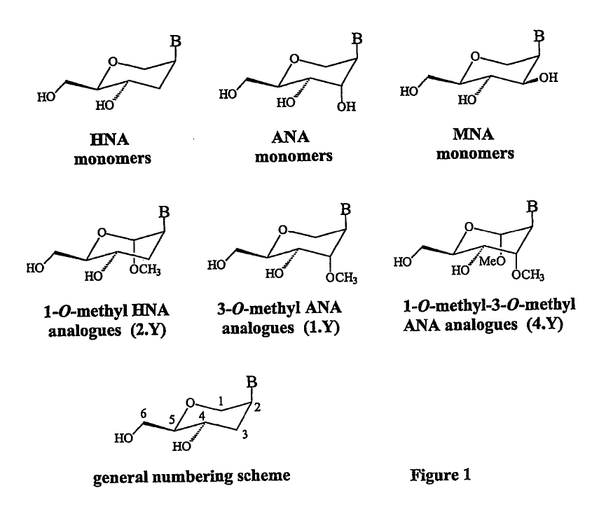

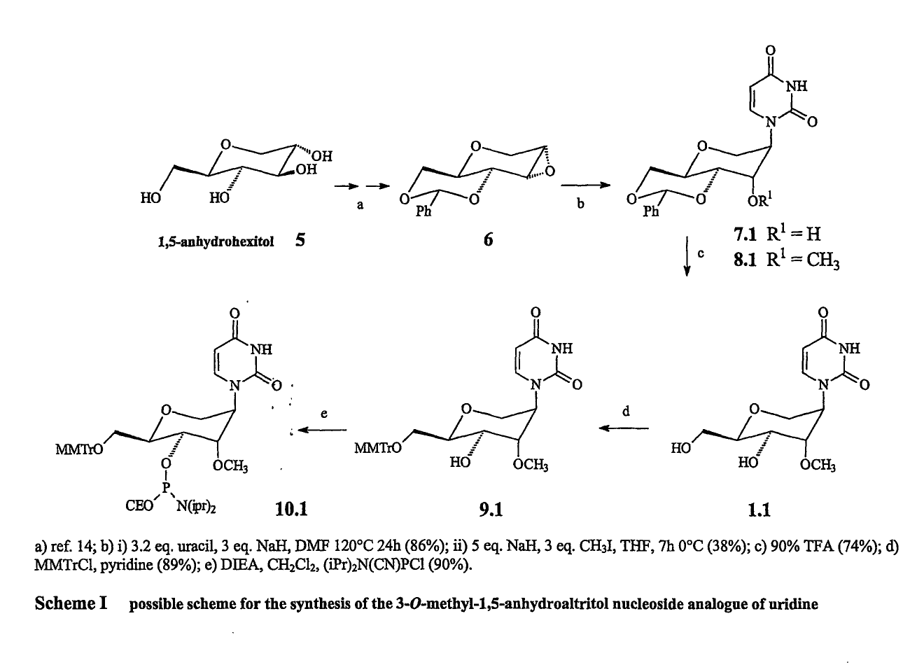

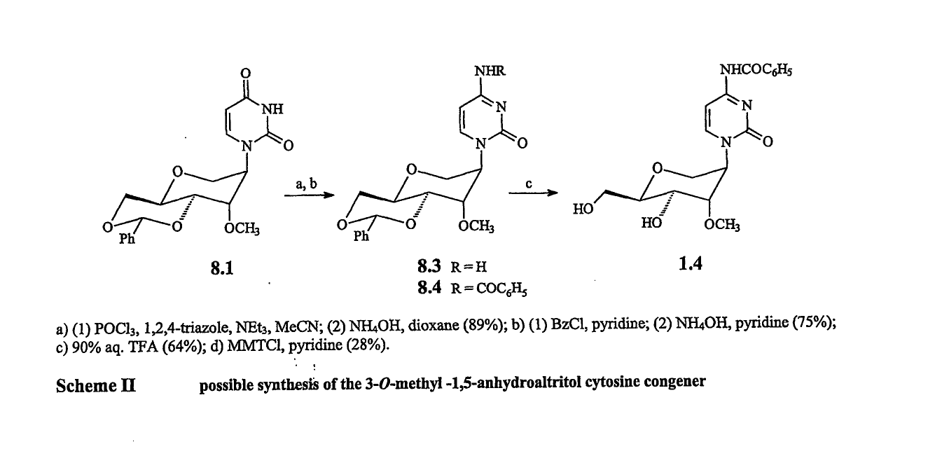

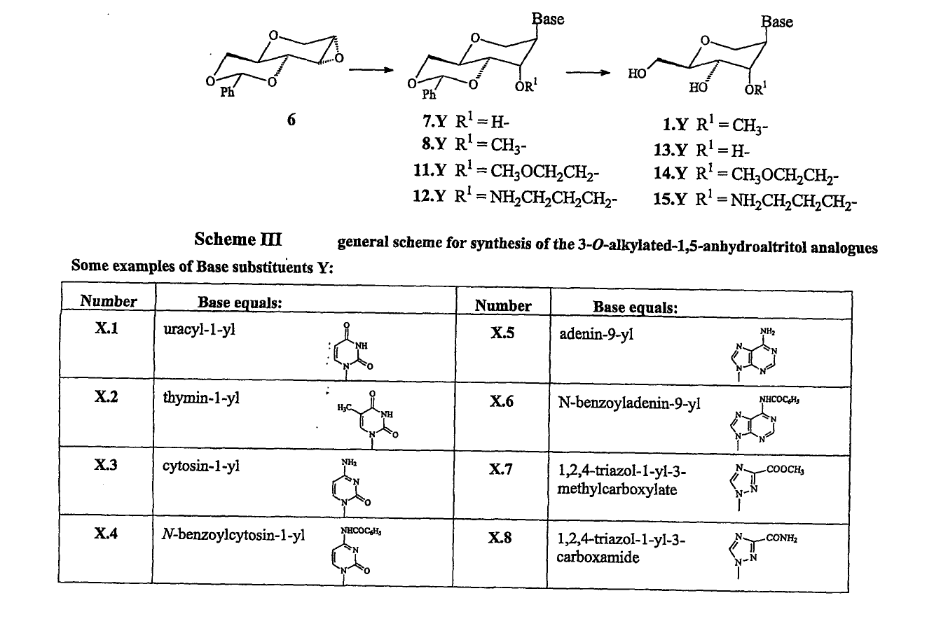

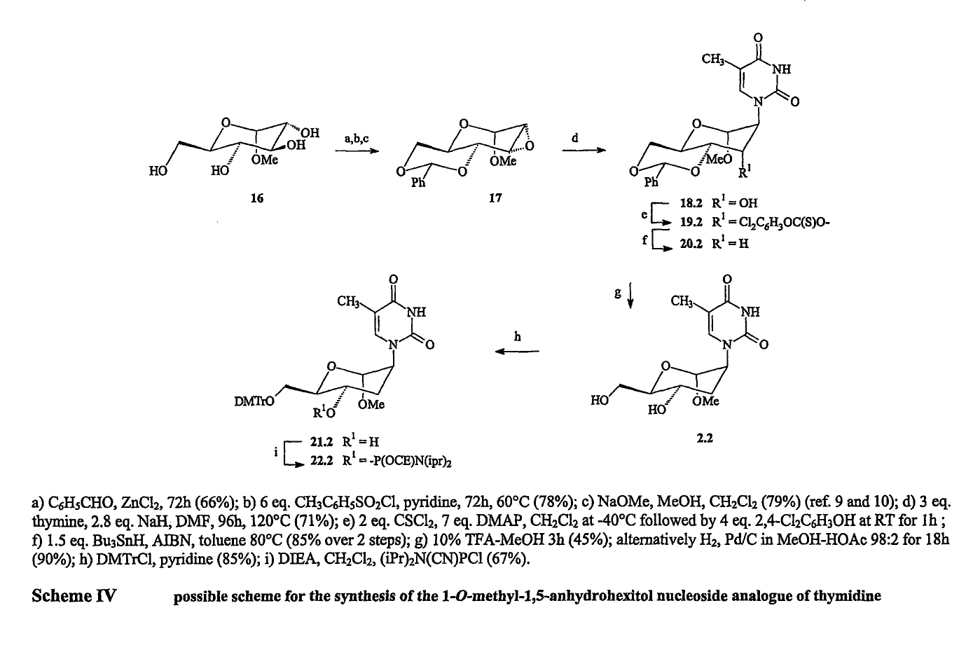

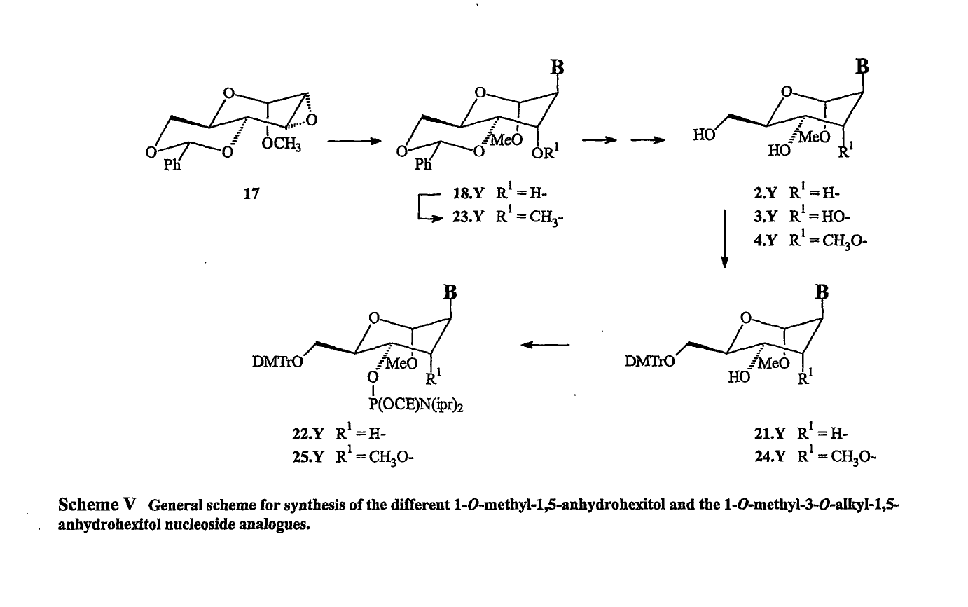

An interesting observation made during hybridization experiments is that the HNA:RNA duplex is invariably more stable than the HNA:DNA duplex. Molecular dynamics simulation of HNA:RNA and HNA:DNA hybrids revealed that minor groove solvatation contributes to this difference in duplex stability ([11] De Winter et al., J. Am. Chem. Soc. 1998, 120, 538 1). In order to further increase minor groove hydration, in an effort to influence hybridization in a beneficial way, we synthesized D-altritol nucleic acids (ANA), consisting of a phosphorylated D-altritol backbone with nucleobases inserted in the 2′-position of the carbohydrate moiety ([12] Allart et al., Chemistry: European J., 1999, 5, 2424) (see FIG. 1). They differ, structurally, from HNA as described in references [1-4] by the presence of a supplementary hydroxyl group in the 3′-α-position, meaning that carbon-3′ of the hexitol moiety adopts the [S]-configuration. Inversion of configuration, giving the 3′-[R]-form leads to D-mannitol nucleic acids (MNA) which lack hybridization capabilities with natural nucleic acids ([13] Hossain et al., J. Org. Chem. 1998, 63, 1574). This is due to conformational restriction of single stranded MNA in a partially unwound form by formation of intrastrand hydrogen bonds between the 3′-hydroxy and the 6′-O of the phosphate of the next nucleotide. This hydrogen bond, however, cannot be formed when using 2-deoxy-1,5-anhydro-D-altritol nucleosides as repeating unit in the backbone structure (ANA). The 3′-hydroxyl group of this nucleoside analogue is pointing into the minor groove of the ANA:RNA duplex and positively influences hybridization either by increasing hydration of the groove either by further stabilization of a preorganized single stranded structure (see reference [12]). The higher thermal stability for ANA:RNA was demonstrated when compared with HNA:RNA duplexes. Complexes formed between ANA and natural nucleic acids are, likewise, more stable than complexes between two natural nucleic acid strands.

Moreover, as well the HNA complexes, as the ANA complexes retain their sequence-selectivity as well for polypurine sequences as for completely mixed sequences. In addition it has been shown that oligomers comprising these hexitol modifications are endowed with strong nuclease resistance (see references [2], [3] and [12].

However, the technical difficulty of the latter altritol nucleic acid monomers is the lenghty synthesis and the need of a supplementary protecting group for the 3′-hydroxyl position during oligomer assembly. (see reference [12] and [14] Allart et al., Tetrahedron, 1999, 55, 6527). The HNA monomers themselves, likewise obviate a long-routed synthesis in which twice a deoxygenation step is needed, when starting from ubiquitous glucose (as described in references [15] Verheggen et al., J. Med. Chem., 1993, 36, 2033; [16] Andersen et al., Tetrahedron Lett. 1996, 37, 8147; and [17] De Bouvere et al., Liebigs Ann./Receuil, 1997, 1453). Therefore, constructs endowed with a further increase in affinity for an RNA taget, or constructs constituting a technically more viable alternative for the HNA or ANA monomers would be advantageous.

Reviews on the subject matter and background of antisense oligonucleotides are numerous (see for example the references [8] and [18] Herdewijn, P. Biochem. Biophys. Acta 1999, 1489, 167; [19] Uhlman, E. and Peyman, A. Chem. Rev. 1990, 90, 543; or [20] Carbohydrate Modifications in Antisense Research, ACS Symposium Series 580 (lds.: Sanghvi, Y. S.; Cook, P. D.), Washington, 1994).

In addition, the beneficial effects of 2′-O-methylribonucleosides (as described for example in [21] Inoue et al., Nucleic Acids Res. 1987, 15, 6131; and [22] Sproat et al., Nucleic Acids Res., 1989, 17, 3373), or of 2′-O-methoxyethylnbonucleosides (and 2′-O-alkylribonucleosides in general) (see [23] P. Martin, Helv. Chim. Acta 1995, 78, 486), or of 2′-O-aminopropylribonucleosides (see references [23] and [24] Griffey et al., J. Med. Chem. 1996, 39, 5100), and of 2′-O-(dialkyl)aminooxyethyl ribonucleosides (as described in [25] Prakash et al., Org. Lett., 2000, 2, 3995) have been documented before and further references to these derivatives can be found in the different reviews quoted (references [8], [18], [19] and [20]). Only a few references are given here as examples, and all are related to ribofuranose modifications.

In addition, patent applications have been filed describing different carbohydrate modifications of natural oligoribonucleotides for use in oligomers. WO 00/03720 describes the use of carbohydrate or 2′-modified oligonucleotides having alternating internucleoside linkages and modulating the activity of wild-type nucleic acids. In addition, WO 00/08042 details the use of aminooxy-modified nucleosidic compounds and oligomeric compounds prepared therefrom for increasing binding affinity to complementary strands. Both series of modifications are limited to the ribofuranose series of analogues.

Methylation of the sugar moiety of either natural nucleosides or nucleoside analogues has been reported many times (see among others, references [21], [22] and the more recent work of [26] Pfundheller et al., Nucleosides Nucleotides, 1999, 18, 2017; and of [27] Wang et al., Tetrahedron Lett., 1996, 37, 6515).

Heterocyclic bases can be converted into one another, and this practical advantage has been used many times for converting uracil nucleoside analogues into cytosine congeners as exemplified by Lin ([28] Lin et al., J. Med. Chem., 1983, 26, 1691).

Many base modifications of the natural pyrimidine and purine rings (as there are thymine, uracil, cytosine, adenine and guanine) among which the diaminopurine ring, the use of a xanthine base or of a 5-propynylated pyrimidine, among many others have been described for improving the hybridisation potential of oligonucleotides and are based on natural nucleosides with the ribofuranose or 2′-deoxyribofuranose skeleton. These modifications have been reviewed for example by Herdewijn ([29] Luyten, I. and Herdewijn, P. Eur. J. Med. Chem., 1998, 33, 515; [30] Herdewijn, P. Antisense & Nucleic Acid Drug Dev., 2000, 10, 297).

Likewise, other heterocyclic bases have been introduced on the (deoxy)ribofuranose skeleton in view of their possible use as universal DNA base analogues as recently reviewed by David Loakes for the deoxyribonucleotide series ([31] Loakes, D. Nucleic Acids Res., 2001, 29, 2437). Most interesting base analogues comprise the nitroazole base analogues, among which the 5-nitroindole, and the azole carboxamides among which the 1,2,4-triazole-3-carboxamide.

Different hexopyranose sugar derivatives have been prepared in the past and the epoxide of methyl 2,3-anhydro-allo-hexopyranoside has been used for synthesis of methyl altroside derivatives by epoxide ring opening reactions ([32] Richtmeyer et al., J. Am. Chem. Soc., 1941, 63, 1727; [33] Rosenfeld et al., J. Am. Chem. Soc., 1948, 70, 2201).

Assembly of oligonucleotides generally and preferably takes place on solid support, and commercial supports are available containing one of the natural bases already attached to the solid support material via a cleavable linker. Supports are chosen depending on the 3′-penultimate base of the desired sequence. New nucleoside analogues require synthesis of supports loaded with the modification to allow for synthesis of oligomers with the analogue at the 3′-penultimate end. In addition, universal supports have been described generating a 3′-phosphate at the 3′-end (commercially available, e.g. Glen Research) or leaving a small organic residue at the 3′-end which does not interfere with hybridisation. As an example, oligos can be assembled on a propanediol containing universal support ([34] Van Aerschot et al., Bull. Soc. Chim. Belges, 1995, 104, 717). More recently, universal supports became available commercially, leaving no trace and thus generating true 3′-hydroxyl ends and which therefore can be considered true universal supports (“novel universal supports featuring rapid amide assisted dephosphorylation”, Glen Research).

Several publications of Walder et al. describe the interaction of RNase H and oligonucleotides. Of particular interest are: [35] Dagle et al., Nucleic Acids Res. 1990, 18, 4751; [36] Dagle et al., Antisense Res. and Dev. 1991, 1, 11; [37] Eder et al., J. Biol. Chem. 1991, 266, 6472; and [38) Dagle et al., Nucleic Acids Res. 1991, 19, 1805. According to these publications DNA oligonucleotides having both unmodified phosphodiester internucleotide linkages and modified phosphorothioate internucleoside linkages are substrates for cellular RNase H. Since they are substrates, they activate the cleavage of target RNA by RNase H. However, the authors further note that in Xenopus embryos, both phosphodiester linkages and phosphorothioate linkages are also subject to exonuclease degradation. Such nuclease degradation is detrimental since it rapidly depletes the oligonucleotide available for RNase H activation.

As described in references [35], [36], and [38], to stabilize oligonucleotides against nuclease degradation while still providing for RNase H activation, 2′-deoxy oligonucleotides having a short section of phosphodiester linked nucleotides positioned between sections of phosphoramidate, alkyl phosphonate or phosphotriester linkages were constructed.

U.S. Pat. No. 5,149,797, issued Sep. 22, 1992, discloses mixed phosphate backbone oligonucleotides which include an internal portion of deoxynucleotides linked by phosphodiester linkages, and flanked on each side by a portion of modified DNA or RNA sequences. The flanking sequences include methyl phosphonate, phosphoromorpholidate, phosphoropiperazidate or phosphoroamidate linkages.

U.S. Pat. No. 5,256,775, issued Oct. 26, 1993, describes mixed oligonucleotides which incorporate phosphoroamidate linkages and phosphorothioate or phosphorodithioate linkages. Use of phosphorothioate linkages for the internal portion of deoxynucleotides to be recognized by RNase H indeed stabilizes the oligomer against nuclease degradation.

Hexitol nucleic acids likewise have been combined with a deoxynucleotide window comprising phosporothioate linkages, to activate RNase H in an effort to increase the biological effect of modified nucleic acids with improved hybridisation potential (see reference [9]).

Although it has been recognized that cleavage of a target RNA strand using an oligonucleotide and RNase H would be useful, nuclease resistance of the oligonucleotide and fidelity of hybridisation are of great importance in the development of oligonucleotide therapeutics. Accordingly, there remains a need for methods and materials that can activate RNase H while concurrently maintaining or improving hybridisation properties and providing nuclease resistance. Such oligonucleotides are also desired as research reagents and diagnostic agents.

### SUMMARY OF THE INVENTION

The present invention is directed to nucleoside analogues with as substitute for the sugar part a 1,5-anhydrohexitol moiety, deoxygenated and substituted with a nucleobase at the 2-position, of which the hexitol ring is further substituted with at least one alkoxy substituent at the 3-position or at the 1-position, and to oligonucleotides wherein at least some of the nucleotides are part of the afore mentioned hexitol nucleoside analogues and which exhibit sequence-specific hybridization to complementary sequences of nucleic acids, and maintaining or improving the hybridisation strength. The invention further relates to nucleoside analogues with a 1,5-anhydrohexitol moiety as the sugar part deoxygenated and substituted with a nucleobase at the 2-position, of which the hexitol ring is substituted with a methoxy substituent at the 1-position, having at the same time either a hydroxy or an alkoxy group at the 3-position, or having a 3-deoxygenated position. The inclusion of one or more of the afore mentioned hexitol nucleoside analogues in oligonucleotides provides, inter alia, either for improved binding or for maintained binding of these oligonucleotides to a complementary strand. This invention further relates to the chemical synthesis of these oligomers which are useful for diagnostics, therapeutics and as research agents.

### DESCRIPTION OF THE ILLUSTRATIVE EMBODIMENTS

To further augment the affinity for target RNA structures, two possible strategies which can be explored are a search for analogues which either increase the conformational preorganisation of the monomeric structures, or which alternatively augment the hydrophobic interactions. In addition, technical ease of synthesis needs to be considered.

Hereto, alkylated hexitol nucleoside analogues have been developed, with alkylation carried out either at the C1 position or at the C3 position or at both positions concomitantly. As an example therefore, 3′-O-alkylated, and in particular but not limiting 3′-O-methylated altrohexitol nucleoside analogues; have been prepared. As further examples, 1′-O-methylated altrohexitol nucleoside analogues, the latter alternatively named methyl altropyranoside nucleoside analogues, were prepared. Still further examples will be given describing methylation at both C1 and C3, affording 1′,3′-bis-O-methyl altrohexitol nucleoside analogues. All afore mentioned analogues successfully can be incorporated into oligomers either as homopolymers or as analogues comprised, individually or in stretches, within natural oligomers or known oligomer analogues, like hexitol nucleic acids.

The 3′-O-ailcylated altrohexitol monomers preferably can be synthesized analogous to the preparation of the altrohexitol monomers (see reference [14]), with 3′-O-alkylation of the pre-formed nucleoside analogue. The 1′-O-methylated monomers preferably can be synthesized using the ubiquitous methyl glucopyranoside as starting material. Attachment of the heterocyclic base in this series can be envisaged preferably according to an analogous strategy as for synthsis of the 3′-O-alkylated analogues, via ring opening of the allopyranoside epoxide 17 (Scheme IV, vide infra). Further derivatisation can allow either deoxygenation of the 3′-position or alkylation of the 3′-position in analogy with the afore mentioned 3′-O-alkylated altrohexitol nucleoside analogues or protection of the remaining 3′-hydroxyl as in altritol nucleic acids (see ref [12]) to allow for 1′-O-methyl altritol nucleic acids.

Assembly of monomers into an oligomer can be done according to classical schemes and can be carried out either by standard phosphoramidite chemistry (compare [39] Matteucci and Caruthers, J. Am. Chem. Soc. 1981, 103, 3185) or by phosphonate chemistry (compare [40] Froehler et al., Nucleic Acids Res. 1986, 14, 5399). All procedures are conveniently carried out on an automated DNA synthesizer as for standard oligonucleotide synthesis. For these standard conditions also compare for example reference [41] Methods in Molecular Biology, vol. 20, Protocols for oligonucleotides and analogues, S. Agrawal ed. The preferred method is the phosphoramidite method making use of the phosphoramidites of the hexitol nucleoside analogues as the incoming building blocks for assembly in the “6′-direction”.

Functionalization of the monomers comprising base protection, primary hydroxyl group protection and phosphitylation likewise can be carried out according to traditional schemes (see also reference [41]). Base protection and tritylation of the primary hydroxyl group can be combined in one reaction as described by [42] Ti et al., J. Am. Chem. Soc. 1982, 104, 1316.

Further examples will be given in which representatives of the afore mentioned nucleoside analogues according to traditional phosphoramidite chemistry strategies, have been converted into their respective phosphoramidites. In addition, examples of all types of differently alkylated altritol nucleoside analogues, 3′-O-alkylated altrohexitol nucleoside analogues, as well as 1′-O-methylated altrohexitol nucleoside analogues, as well as 1′-O-methyl-3′-O-alkyl altrohexitol nucleoside analogues have been incorporated with good yield into oligonucleotides within homopolymers, as well as within natural oligonucleotides, as well as within oligomer analogues, exemplified by hexitol nucleic acids.

Thermal denaturation experiments indicate strong duplex stabilities for the new analogues with either improved or maintained hybridisation potential, as well for hybridisation with RNA targets, as for pairing with hexitol nucleic acids.

For reasons of clarity, a general figure has been inserted, depicting the different hexitol containing nucleoside analogues. These comprise the known 1,5-anhydrohexitol or HNA monomers (Hexitol Nucleic Acids), the known 1,5-anhydroaltritol or ANA monomers (Altritol Nucleic Acids) and the known 1,5-anhydromannitol or MNA monomers (Mannitol Nucleic Acids). In addition, the new structures are depicted with the 1′-O-methylated HNA analogues 2.Y, the 3′-O-alkylated ANA analogues, exemplified by the 3′-O-methylated ANA analogues 1.Y, and the 1′-O-methyl-3′-O-alkyl ANA analogues, exemplified by the 1′-O-methyl-3′-O-methylated ANA analogues 4.Y. (FIG. 1).

A possible synthesis scheme for the 3′-O-methylated hexitol nucleoside analogue of uridine 1.1 is depicted in scheme I, and follows the route previously described for preparation of the altrohexitol monomers (ANA, see reference [14]). Ring opening of the 4,6-O-benzylidene protected allitol epoxide 6 with the uracil anion furnishes the altrohexitol derivative 7.1. Chemoselective methylation without temporary protection of the nucleobase gives the methylated nucleoside 8.1. The methylation proceeds slowly and the yield is lower than reported for other derivatives (see references [26] and [27]). The slow reaction compared to the previously described methylations is probably caused by the axial location of the hydroxyl group. The selectivity of the methylation can be confirmed by NMR, and only a small amount of the 3′-O,N³-dimethylated compound is obtained. Removal of the benzylidene protecting group can be accomplished either through hydrogenation or under acidic conditions furnishing 1.1. Further functionalization to allow oligomer assembly can be done according to various strategies. Phosphoramidite chemistry is one of the preferred strategies and hereto the monomeric compound is functionalized via either dimethoxytritylation or monomethoxytritylation followed by phosphitylation yielding the desired phosphitylated building block 10.1, which can be used for oligomer assembly.

The cytosine congener 1.4 can be obtained from the uracil analogue 8.1 according to well-known procedures (scheme II, see reference [28]). Reaction with POCl₃ and 1,2,4-triazole followed by treatment with aqueous NH₃ affords the 3′-O-methylated cytidine nucleoside analogue 8.3. When using anhydrous pyridine as solvent for the reaction with POCl₃ and 1,2,4-triazole, followed by treatment with ammonia as previously described (see ref [14]), the cytosine nucleoside can be obtained as a yellow substance in only 20% yield. However, by changing the solvent to anhydrous acetonitrile, the cytosine nucleoside 8.4 can be obtained in 89% as a white substance. Benzoylation of the exocyclic aminogroup is followed by acidic hydrolysis to give the parent cytosine nucleoside derivative 1.4. Further functionalization according to traditional strategies can allow introduction into oligomers.

Other heterocyclic bases can be introduced on the same scaffold and other alkyl groups can be attached at the 3-position according to the same strategy as outlined for introduction of the uracil moiety. Further functionalization can allow incorporation of these new 3′-O-alkylated altritol nucleoside analogues into oligomers. Some further examples are given in scheme III not to limit the present invention but to demonstrate the feasability of these general principles. As an example, not to limit the invention, adenine can be introduced according to the same strategy as used for introduction of the heterocyclic base uracil, providing the analogue 7.5, and further modification can lead for example to the compounds 7.6, 8.5 and 8.6.

Analogously, unnatural heterocyclic bases can be attached to the here described modified hexitol rings and thus can be incorporated into oligomers. Such analogues as for example modified monomers containing as the heterocyclic moiety a diaminopurine, a xanthine or a 5-propynylated pyrimidine, among many others, can lead to further increases in hybridisation potential, as in other series of nucleoside modifications, as reviewed for example by Herdewijn (see references [29] and [30]). Likewise, other heterocyclic bases can be introduced and further functionalized according to the same strategies in view of their possible use as universal DNA base analogues as recently reviewed by David Loakes for the deoxyribonucleotide series (see reference [31]). Possible base analogues which can be envisaged therefore are nitroazole base analogues, among which the 5-nitroindole, and the azole carboxamides among which the 1,2,4-triazole-3-carboxamide. The latter heterocycle can for instance be introduced via the 1,2,4-triazole-3-methylcarboxylate affording the analogue 7.7, which can be further fictionalized according to the previously outlined strategies to the hexitol analogues 7.8, 8.7, 8.8, 13.8 or 1.8, respectively.

The 1′-O-methylated HNA analogues (schemes IV and V) can be obtained starting from ubiquitous methyl glucopyranoside (16) and the general procedure is exemplified for the thymine analogue 2.2 and its functionalized phosphoramidite 22.2. According to this route, opening of the epoxide ring of methyl 2,3-anhydro-allo-hexopyranoside 17 (see references [32] and [33]) with different heterocyclic bases affords the compounds 18.Y, which can be followed by deoxygenation of the 3-position, affording the 3′-deoxy analogues 20.Y. Removal of the benzylidene position with acid is possible, but less straightforward because of the glycosidic linkage, but can be accomplished alternatively via hydrogenation in almost quantitative yield, affording the envisaged 1′-O-methylated analogues 2.Y of 1,5-anhydrohexitol nucleosides. Further modification yields the desired phosphitylated building blocks 22.Y, to be used for oligomer assembly.

Alternatively, alkylation procedures as outlined for the synthesis of the analogues 1.Y, can be used to obtain the analogues 4.Y as depicted in scheme V. Synthesis of the required phosphoramidite building blocks can be done according to well-known strategies providing the phosphoramidite analogue 25.Y. This is exemplified further by synthesis of the thymine and adenine nucleoside analogues 25.2 and 25.6, respectively, which both as examples are used for oligomer assembly. Following identical strategies, other 3′-O-alkyl groups likewise can be introduced, resulting in different 1′-O-methyl-3′-O-alkyl altritol nucleoside analogues, which can be incorporated into oligomers following conversion to for example phosphoramidite building blocks.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLE 1

### 3′-O-methylation

1,5-anhydro-4,6-O-benzylidene-3-O-methyl-2-(uracil-1-yl)-2-deoxy-D-altro-hexitol (8.1).

1,5-anhydro-4,6-O-benzylidene-2-(uracil-1-yl)-2-deoxy-D-altro-hexitol (7.1) (see reference [14]; 1.59 g, 4.6 mmol) was coevaporated with anhydrous acetonitrile (3×10 mL) and dissolved in anhydrous THF (40 mL). NaH (552 mg, 13.8 mmol) was added, and the reaction was left to stir for 30 min at 0° C., whereupon CH₃I (1.35 mL, 23 mmol) was added. After 5 hours stirring at 0° C. an additional amount of CH₃I (1 mL, 17 mmol) was added, and the reaction was left to stir another 2 hours at 0° C. The reaction was quenched with water (20 mL), diluted with EtOAc (200 mL) and washed with NaHCO₃ (2×50 mL). The combined aqueous phase was extracted with dichloromethane (50 mL), whereupon the combined organic phase was dried (Na₂SO₄), filtered and evaporated to dryness. Purification by silica column chromatography (0-2% MeOH/dichloromethane) afforded the methylated nucleoside 8.1 (829 mg, 2.28 mmol, 50% (69% based on recovered starting material)) as a white foam. R_{f}: 0.3 (5% MeOH/dichloromethane).

δ ¹H-NMR (CDCl₃): 9.69 (s, 1H, NH), 8.04 (d, J=8.1 Hz, 1H, 6-H), 7.34-7.49 (m, 5H, Ph), 5.80 (d, J=8.1 Hz, 1H, 5-H), 5.30 (s, 1H, PhCH), 4.53 (t, J=2.9 Hz, 1H, 2′-H), 4.37 (dd, J=5.5, 9.9 Hz, 1H, 6′-Hₑ), 4.32 (dd, J=3.3, 13.2 Hz, 1H, 1′-H.), 4.08 (dt, J=5.1, 9.9 Hz, 1H, H-5′), 4.03 (d, J=13.9 Hz, 1H, 1′-Hₐ), 3.86 (br t, 1H, 3′-H), 3.81 (d, J=10.3 Hz, 1H, 6′-Hₐ), 3.64 (dd, J=2.6, 9.5 Hz, 1H, 4′-H), 3.63 (s, 3H, OCH₃). δ ¹³C-NMR (CDCl₃): 163.30 (C4), 150.79 (C2), 142.05 (C6), 137.01, 129.03, 128.15, 126.00 (Ph), 102.60 (C5), 102.23 (PhCH), 76.22 (C4′), 74.58 (C3′), 68.70 (C6′), 66.45 (C5′), 64.11 (C1′), 59.41 (OCH₃), 54.64 (C2′). HRMS (thgly) calcd. for C₁₈H₂₀N₂NaO₆ [M+Na]⁺: 383.1219, found 383.1229.

### EXAMPLE 2

### Benzylidene Cleavage

1,5-anhydro-3-O-methyl-2-(uracil-1-yl)-2-deoxy-altro-hexitol (1.1).

1,5-anhydro-4,6-O-benzylidene-3-O-methyl-2-uracil-1-yl)-2-deoxy-D-altro-hexitol (8.1) (390 mg, 1.08 mmol) was dissolved in 90% aq. trifluoroacetic acid (6 mL) and stirred at room temperature for 1 hour. Upon completion, the mixture was evaporated to dryness and coevaporated with toluene (2×10 mL). Purification by silica column chromatography (5-10% MeOH in dichloromethane) afforded the deprotected nucleoside 1.1 as a white foam (210 mg, 0.77 mmol, 71%). R_{f}: 0.28 (10% MeOH/dichloromethane).

δ ¹H-NMR (DMSO-d6): 11.32 (s, 1H, NH), 7.98 (d, J=8.06 Hz, 1H, 6-H), 5.57 (dd, J=2.2, 8.06 Hz, 1H, 5-H), 4.85 (d, J=6.23 Hz, 1H, 4′-OH), 4.60 (t, J=5.86 Hz, 1H, 6′-OH), 4.46 (AB, J=3.66, 1H, 2′-H), 3.86 (d, J=3.66 Hz, 2H, 1′-H), 3.51-3.68 (m, 5H, 3′-H, 4′-H, 5′-H, 6′-H), 3.39 (s, 3H, OCH₃). δ ¹³C-NMR (DMSO-d6): 163.42 (C-4), 151.31 (C-2), 143.27 (C-6), 101.35 (C-5), 78.01, 77.23 (C-3′ and C-4′), 63.60 (C-5′), 63.08 (C-1′), 60.14 (C-6′), 57.62 (OCH₃), 52.77 (C-2′). HRMS (thgly) calc. for C₁₁H₁₅N₂Na₂O₆ (M—H+2Na)⁺: 317.07255, found 317.07232.

### EXAMPLE 3

### 6′-O-protection

1,5-anhydro-3-O-methyl-6-O-monomethoxytrityl-2-(uracil-1-yl)-2-deoxy-D-altro-hexitol (9.1).

1,5-anhydro-3-O-methyl-2-(uracil-1-yl)-2-deoxy-D-altro-hexitol (1.1) (460 mg, 1.69 mmol) was coevaporated with anhydrous pyridine (2×5 mL) and redissolved in anhydrous pyridine (10 mL). Monomethoxytrityl chloride (532 mg, 1.73 mg) was added, and the reaction was left to stir for 20 hours. After completion, the reaction was quenched with methanol (2 mL) and evaporated to dryness. The last residues of pyridine were removed by coevaporation with toluene. Purification by silica column chromatography (1-5% MeOH/dichloromethane) afforded the tritylated compound as a white foam (816 mg, 1.50 mmol, 89%). R_{f}: 0.79 (5% MeOH/dichloromethane). The reaction alternatively can be carried out using dimethoxytrityl chloride.

δ ¹H-NMR (DMSO-d₆): 11.40 (s, 1H, NEW, 8.06 (d, 3=8.1 Hz, 6-H), 6.88-7.43 (m, 14H, MMTr), 5.58 (d, J=8.1 Hz, 1H, 5-H), 4.80 (d, J=6.6 Hz, 1H, 4′-OH), 4.45 (m, 1H, 2′-H), 3.95 (m, 2H, 1H), 3.60-3.86 (m, 5H, MMTr-OCH₃, 4′-H, 5′-H), 3.54 (t, J=3.7 Hz, 1H, 3′-H), 3.41 (s, 3H, OCH₃), 3.21 (d, J=2.6 Hz, 2H, 6′-H). δ ¹³C-NMR (DMSO-d₆): 163.41 (C4), 151.21 (C2), 143.08 (C6), 158.41, 144.75, 135.28, 127.02-130.36, 113.33 e r), 101.41 (C5), 85.56 (MMTr), 77.37 (C5′), 75.91 (C3′), 63.80 (C4′), 63.25 (C1′), 62.25 (C6′), 58.00 (OCH₃), 55.15 (er), 52.78 (C2′). HRMS (thgly) calcd. for C₃₁H₃₂N₂NaO₇ [M+Na]⁺: 567.2107, found 567.1817.

### EXAMPLE 4

### Phosphitylation

1,5-anhydro-3-O-methyl-4-O-(P-βcyanoethyl-N,N-diisopropylaminophosphinyl)-6-O-monomethoxytrityl-2-(uracil-1-yl)-2-deoxy-D-altro-hexitol (10.1).

The monomethoxytritylated derivative 9.1 (495 mg, 0.90 mmol) was dissolved in 6 mL dichloromethane under argon and diisopropylethylamine (470 μL, 2.70 mmol) and 2-cyanoethyl N,N-diisopropylchlorophosphoramidite (305 μL, 1.35 mmol) were added and the solution was stirred for 2 hours. An additional amount of 1.35 mmol DIPEA and 0.65 mmol of the amidite were added and the mixture was stirred for another 2 hours TLC indicated complete reaction. Water (3 mL) was added, the solution was stirred for 10 min. and partitioned between CH₂Cl₂ (50 mL) and aqueous NaHCO₃ (30 mL). The organic phase was washed with aqueous sodium chloride (2×30 mL) and the aqueous phases were back extracted with CH₂Cl₂ (30 mL). Evaporation of the organics left an oil which was flash purified twice on 40 g of silica gel (hexane: acetone: TEA, 49:49:2) to afford the product as a foam after coevaporation with dichloromethane. Dissolution in 2 mL of dichloromethane and precipitation in 60 mL cold (−70° C.) hexane afforded 605 mg (0.81 mmol, 90%) of the title product 10.1 as a white powder. Rf (hexane: acetone: TEA 49:49:2): 0.32.

ESI-MS pos. calcd. for C₄₀H₅₀N₄O₈P₁ 745.33660 found 745.3429 [M+H]⁺; ³¹P-NMR δ (ppm, external ref.=H₃PO₄ capil.) 148.11, 150.40.

### EXAMPLE 5

### Uracil to Cytosine Conversion

1,5-anhydro-4,6_O-benzylidene-3-O-methyl-2-(cytosin-1-yl)-2-deoxy-D-altro-hexitol (8.3)

To a solution of 1,5-anhydro-4,6-O-benzylidene-3-O-methyl-2-(uracil-1-yl)-2-deoxy-D-altro-hexitol (8.1) (602 mg, 1.67 mmol) in anhydrous acetonitrile (21 mL) was added 1,2,4-triazole (1.08 g, 15.7 mmol) and POCl₃ (0.31 mL, 3.33 mmol). The reaction mixture was cooled to 0° C. and anhydrous triethylamine (2.1 mL, 15.1 mmol) was added and the reaction was left to stir for 18 hours at room temperature. The reaction was quenched with triethylamine (1.38 mL) and water (0.4 mL) and siring was continued for another 10 minutes, before the mixture was evaporated to dryness. The residue was dissolved in ethylacetate (100 mL) and washed with aq. NaHCO₃ (2×10 mL) and water (10 mL). The aqueous phase was extracted with dichloromethane (50 mL) and the combined organic extract was dried (Na₂SO₄), filtered and evaporated to dryness. The residue was dissolved in dioxane (10 mL) and concentrated ammoniumhydroxide (2 mL) was added, and the reaction was left to stir for 3 days, whereupon it was evaporated to dryness. Silica gel column chromatography (3, 5, 10% MeOH/dichloromethane) afforded the cytosine congener 8.3 as a white foam (540 mg, 1.49 mmol, 89%). R_{f}: 0.21 (7% MeOH/dichloromethane).

δ ¹H-NMR (DMSO-d₆): 7.90 (d, J=7.69 Hz, 1H, 6-H), 7.33-7.42 (m, 5H, Ph), 7.19 (br. S, 2H, NH₂), 5.79 (d, J=7.33 Hz, 1H, 5-H), 5.64 (s, 1H, PhCH, 4.47 (m, 1H, 2′-H), 4.22 (dd, J=4.39, 9.52 Hz, 1H, 6′-Hₑ), 4.09 (s, 2H, 1′-H), 3.81 (m, 1H, 5′-H), 3.63-3.73 (m, 3H, 3′H, 4′-H and 6′-Hₐ), 3.49 (s, 3H, OCH₃). δ ¹³C-NMR (DMSO-d₆): 165.86 (C-4), 155.54 (C-2), 143.37 (C-6), 137.94 (Ph), 129.05, 128.26 and 126.37 (Ph), 101.27 (PhCH), 94.29 (C-5), 76.34 (C-4′), 74.81 (C-3′), 68.25(C-6′), 66.28(C-5′), 64.25(C-1′), 58.45 (OCH₃), 54.08 (C-2′). HRMS (thgly) calc. for C₁₈H₂₂N₃O₅ (M+H)⁺: 360.1559, found 360.1572.

### EXAMPLE 6

### Cytosine Base Protection

1,5-anhydro-4,6-O-benzylidene-3-O-methyl-2-(N⁴-benzoylcytosin-1-yl)-2-deoxy-D-altro-hexitol (8.4).

To a solution of 8.3 (499 mg, 1.39 mmol) in anhydrous pyridine (8 mL) was added benzoylchloride (0.8 mL, 6.9 mmol) at 0° C., and stirring was continued at room temperature for 3 hours. The reaction mixture was cooled to 0° C. and water (1.6 mL) was added, and after 5 min. concentrated ammoniumhydroxide (3.2 mL) was added. Stirring was continued for 30 mL, whereupon the reaction mixture was evaporated to dryness. Purification by silica gel column chromatography (0-5% MeOH/dichloromethane) afforded the benzoylated nucleoside 8.4 as a white foam (480 mg, 1.04 mmol, 75%). R_{f}: 0.74 (7% MeOH/dichloromethane).

δ ¹H-NMR (DMSO-d6): 8.41 (d, J=7.69 Hz, 1H, 6-H), 8.03 (d, J=6.96 Hz, 2H, Bz), 7.3-7.68 (n, 11 H, Bz, Ph, 5-H), 4.61 (br s, 1H, 2′-H), 4.104.28 (m, 3H, 1′-H, 6′-Hₑ), 3.70-3.91 (m, 4H, 3′-H, 4′-H, 6′-Hₐ), 3.53 (s, 3H, OCH₃). δ ¹³C-NMR (DMSO-d6): 168.18, 167.75 (CO), 163.20 (C4), 155.04 (C-2), 147.93 (C-6),126.41-137.93 (2Bz+Ph), 101.31 (PhCH), 96.97 (C-5), 75.91 (C-4′), 74.27 (C-3′), 68.20 (C-6′), 66.44 (C-5′), 64.07 (C-1′), 58.58 (OCH₃), 55.12 (C-2′). HRMS (thgly) calc. for C₂₅H₂₆N₃O₆ (M+H)⁺: 464.1821, found 464.1890.

### EXAMPLE 7

### Benzylidene Cleavage (2)

1,5-anhydro-3-O-methyl-2 (N⁴-benzoylcytosin-1-yl)-2-deoxy-D-altro-hexitol (1.4).

1,5-anhydro-4,6-O-benzylidene-3-O-methyl-2-(N-4-benzoylcytosin-1-yl)-2-deoxy-D-altro-hexitol (8.4) (480 mg, 1.04 mmol) was dissolved in 90% aq. TFA (20 mL) and left to stir at room temperature for 3 hours. Upon completion, the mixture was evaporated to dryness, and silica gel column chromatography (5, 10% MeOH/dichloromethane) afforded the deprotected nucleoside 1A as a pale yellow foam (250 mg, 0.67 mmol, 64%)., R_{f}:0.20 (5% MeOH/dichloromethane).

δ ¹H-NMR (DMSO-d6): 4.49 (d, J=7.5 Hz, 1H, 6-H), 8.01 (d, J=8.5 Hz, 2H, Phₘ), 7.63 (t, J=7 Hz, 1H, Phₚ), 7.53 (t, J=8 Hz, 2H, Phₘ), 7.32 (d, J=7 Hz, 1H, 5-H), 4.63 (d, J=4 Hz, 1H, 2′-H), 4.01 (dAB, J-2.5, 12.5 Hz, 2H, 1′-H), 3.65 (m, 3H, 4′-H, 5′-H, 6′-H_{A}), 3.60 (m, 2H, 3′-H, 6′-H_{B}), 3.46 (s, 3H, OCH₃). δ ¹³C-NMR DMSO-d₆): 167.69 (CO), 162.94 (C4), 155.18 (C-2), 148.32 (C-6), 133.39, 132.94, 128.66 (Ph), 96.40 (C-5), 77.64 (C-5′), 76.82 (C-3′), 63.24 (C-4′), 63.13 (C-1′), 60.23 (C-6′), 57.81 (OCH₃), 54.44 (C-2′); HRMS (thgly) calc. for C₁₈H₂₂N₃O₆ (M+H)⁺: 376.1509, found 376.1499.

### EXAMPLE 8

### Synthesis of 1-O-methyl Hexitol Nucleoside Analogues

Methyl 4,6-O-benzylidene-2-(thymin-1-yl)-2-deoxy-D-altro-hexopyranoside (18.2).

Thymine (3.78 g, 30 mmol) was suspended in 250 ml of anhydrous DMF to which was added 1.13 g of a 60% oil dispersion of sodium hydride (28 mmol) and the mixture was heated on an oil bath for 1 hour at 90° C. The methyl alloside epoxide 17 (see references [32] and [333; 2.64 g, 10 mmol) was added and the mixture was heated for 4 days at 120° C., after which the reaction was cooled, quenched with sodium bicarbonate and concentrated. The residue was partitioned between 200 ml of ethyl acetate and 200 ml of 5% aqueous sodium bicarbonate, and the organics were washed twice with brine. Purification of the organic residue on silica gel (0-2% MeOH/dichloromethane) afforded 2.77 g (7.1 mmol, 71%) of the title compound 18.2 as a foam.

UV (MeOH); λmax 269 nm, ¹H-NMR (CDCl₃) (500 MHz) δ: 1.96 (s, 3H, 5-CH₃), 3.21 (d, 1H, J=3.7 Hz, OH), 3.47 (s, 3H, 1′-OCH₃), 3.71 (dd, 1H, J=3.2 and 9.3 Hz, H4′), 3.82 (t, 1H, J=11.8 Hz, H6′a), 4.15 (m, 1H, H3′), 4.43 (m, 2H, H5′, H6′e), 4.79 (d, 1H, J=2.4 Hz, H2′), 4.85 (s, 1H, H1′), 5.63 (s, 1H, PhCH, 7.32-7.37 and 7.44-7.49 (m, 5H, arom-H), 7.55 (d, J=1.2 Hz, H6), 9.36 (s, 1H, NH); ¹³C-NMR (CDCl₃) δ: 163.45 (C4), 150.65 (C2), 136.85 (C6), 136.81, 129.24, 128.27, 126.18 (Ph), 111.42 (C-5), 102.25 (PhCH), 99.07 (C1′), 75.76 (C4′), 69.06 (C6′), 67.22 (C3′), 58.24 (C5′ and C2′), 55.94 (OCH₃), 12.75 (5-CH₃); ESI-MS pos.: HRMS calcd. for C₁₉H₂₃N₂O₇ [M+H]⁺: 391.1505, found 391.1504.

### EXAMPLE 9

### Deoxygenation Procedure for the 3-Position

Methyl 4,6-O-benzylidene-2-(thymin-1-yl)-2,3-dideoxy-D-arabino-hexopyranoside (20.2).

The methyl 4,6-O-benzylidene-2-thymin-1-yl)-2-deoxy-D-altro-hexopyranoside (18.2) (390 mg, 1 mmol) and 856 mg (7 mmol) of dimethylaminopyridine were dissolved in 15 mL of dry dichloromethane. The reaction mixture was cooled to −40° C., and 0.158 mL (2 mmol) of thiophosgene was added with vigorous stirring. The mixture was brought to room temperature, and after stirring for 1 hour, 656 mg (4 mmol) of 2,4-dichlorophenol was added and stirring was continued for 2 hours more. The mixture was poured into 20 mL of a 1 M solution of KH₂PO₄ and extracted twice with dichloromethane. The organic layers were dried, and following evaporation the residue was purified by flash chromatography (0-2% MeOH/dichloromethane). The obtained product 19.2 [FABMS 595 [M+H]⁺] preferably is used immediately for deoxygenation.

Hereto, the obtained thiocarbonyl compound was dissolved in 15 mL of anhydrous toluene. After nitrogen gas was bubbled through the solution for 10 min., 0.41 mL (1.5 mmol) of tributyltin hydride and 20 mg of 2,2′-azobis(2-methylpropionitrile) were added, and the mixture was heated at 80° C. overnight, when TLC indicated complete reaction. The mixture was evaporated and purified on silica gel (0-2% MeOH/dichloromethane) affording 320 mg (0.85 mmol, 85%) of the title compound 20.2.

UV (MeOH) λmax 269 nm; ¹H-NMR (CDCl₃) δ: 1.98 (d, J=1 Hz, 3H, 5-CH₃), 2.19 (ddd, J_{3′3′}=13.7 Hz, 1H, 3′-Hₑ), 2.30 (ddd, J_{3′3′}=13.2 Hz, J_{3′4′}=12.4 Hz, J_{3′2′}=5.1 Hz, 1H, 3′-Hₐ), 3.45 (s, 3H, 1′-OCH₃), 3.72 (ddd, 1H, 4′-H), 3.81. (t, 3-10.4 Hz, 1H, 6′-Hₐ), 3.96 (dt, 1H, J=9.9 and 4.9 Hz, 5′-H), 4.36 (dd, J=4.8, 10.4 Hz) 1H, 6′-Hₑ), 4.77 (s, 1H, 1′-H), 4.81 (dd, 1H, J=2.4 and 4.9 Hz, 2′-H), 5.58 (s, 1H, PhCH), 7.32-7.47 (m, 5H, arom-H), 7.72 (s, 1H, 6-H), 9.21 (s, 1H, NH); ¹³C-NMR (CDCl₃) δ: 163.53 (C4), 150.75 (C2), 137.27 (C6), 137.09, 129.18, 128.31, 126.07 (Ph), 110.70 (C5), 102.14 (PhCH), 98.42 (C1′), 73.20 (C4′), 69.11 (C6′), 65.08 (C5′), 55.07 (OCH₃), 53.59 (C2′), 29.60 (C3′), 12.79 (5-CH₃); ESI-MS pos.: HRMS calcd. for C₁₉H₂₃N₂O₆ [M+H]⁺: 375.1556, found 375.1556.

### EXAMPLE 10

### Benzylidene Cleavage (3)

Methyl 2-(thymin-1-yl)-2,3-dideoxy-D-arabino-hexopyranoside (2.2).

Method A

An amount of 500 mg (1.33 mmol) of the benzylidene protected compound 20.2 was dissolved in 25 mL of methanol and 2.5 mL of trifluoroacetic acid was added. The solution was stirred for 3 hours, evaporated to dryness and coevaporated twice with dioxane. The residue was dissolved in methanol, adsorbed on silica gel by evaporation, and purified by flash chromatography on silica gel (0-15% MeOH/dichloromethane) to afford the title compound 2.2 in 45% yield (172 mg, 0.6 mmol).

Method B

An amount of 1.08 g (2.89 mmol) of the benzylidene protected compound 20.2 was dissolved in 40 mL of methanol, and 0.5 mL of acetic acid was added. The solution was degassed by bubbling nitrogen for 10 min. after which 450 mg of 10% Pd on carbon was added and the mixture was hydrogenated overnight on a Parr apparatus at 45 psi. The mixture was filtered, the filter was washed with hot ethanol, the volatiles were removed in vacuo and the residue was coevaporated twice with dioxane. Crystallization from hexane afforded the title compound 2.2 in 90% yield (743 mg, 2.60 mmol).

UV (MeOH) λmax 269 nm (ε=9400), λmin 235 nm (ε=1650), λmax 209 nm (ε=8700); ¹H-NMR (500 MHz, DMSO-d₆) δ: 1.76 (s, 3H, 5-CH₃), 1.73-1.81 (m, 1H, He3′), 1.97 (dt, 1H, J=5.4 and 13.7 Hz, Ha3′), 3.30 (s, 3H, 1′-OCH₃), 3.43 (m, 1H, H5′), 3.59-3.67 (dAB, 2H, H6′), 3.73 (ddd, 1H, H4′), 4.51 (ddd, 1H, H2′), 4.75 (t, 1H, 6′-OH), 4.76 (d, J=3.4 Hz, 1H, H1′), 4.93 (d, 1H, J=3.9 Hz, 4′-OH), 7.76 (s, 1H, H6), 11.24 (s, 1H, NH); ¹³C-NMR (DMSO-d₆) δ: 163.68 (C4), 150.96 (C2), 138.48 (C6), 108.73 (C5), 97.73.(C1′), 75.05 (C5′), 60.52 (C4′), 60.24 (C6′), 54.26 (OCH₃), 52.24 (C2′), 32.26 (C3′), 12.21 (5-CH₃); ESI-MS pos.: HRMS calcd. for C₁₂H₁₈N₂O₆Na [M+Na]⁺: 309.1063, found 309.1063

### EXAMPLE 11

### 6′-O-protection (2)

6-O-Dimethoxytrityl-2-(thymin-1-yl)-2,3-dideoxy-D-methylglucopyranoside (21.2).

Following coevaporation with anhydrous pyridine, an amount of 910 mg (3.18 mmol) of the thymine glucopyranoside 2.2 was dissolved in 25 mL of pyridine and dimethoxytrityl chloride (1.19 g, 3.5 mmol) was added. The mixture was stirred for 16 h at ambient temperature, quenched with 3 mL of methanol and neutralized with some aqueous sodium bicarbonate. The mixture was concentrated and partitioned twice between dichloromethane and aqueous sodium bicarbonate. The organic layer was purified on 40 g of silica gel with a methanol step gradient (0 to 1%) in dichloromethane containing 0.5% of pyridine, affording 1600 mg (2.72 mmol, 85%) of the title compound 21.2 as a foam.

¹H-NMR 500 MHz (CDCl₃) δ: 1.82 (s, 3H, 5-CH₃), 2.00-2.07 (ddd, 1H, 3′-Hₐ), 2.12-2.18 (ddd, 1H, 3′-Hₑ), 2.28 (d, 1H, J=3.5 Hz, 4′-OH), 3.38 (s, 3H, 1′-OCH₃), 3.45 (d, J=3.6 Hz, 2H, 6′-H), 3.69 (dt, 1H, J=9 and 8.5 Hz, 5′-H), 3.78 (s, 6H, 2×OCH₃), 3.95 (m, 1H, 4′-H), 4.70 (t, 1H, J=6.5 Hz, 2′-H), 4.75 (s, 1H, 1′-H), 6.84 (2d, 4H, J=9 Hz, arom-H), 7.20-7.47 (m, 9H, arom-H), 7.74 (d, J=1.1 Hz, 6-H), 9.05 (s, 1H, NH); ¹³C-NMR (CDCl₃) δ: 163.59 (C4), 150.88 (C2), 137.98 (C6), 110.51 (C5), 98.14 (C1′), 86.69 (Ph₃C), 72.33 (C5′), 63.54 (C4′), 63.05 (C6′), 55.20 (2×CH₃O), 54.91 (1′-OCH₃), 53.50 (C2′), 31.95 (C3′), 12.65 (5-CH₃)+aromatic signals; ESI-MS pos.: HRMS calcd. for C₃₃H₃₆N₂O₈Na 611.2369; found 611.2364 [M+Na]⁺.

### EXAMPLE 12

### Phosphitylation (2)

6-O-Dimethoxytrityl-2-(thymin-1-yl)-4-O-(P-β-cyanoethyl-N,N-diisopropylamino-phosphinyl)-2,3-dideoxy-β-D-methylglucopyranoside (22.2).

The dimethoxytritylated derivative 21.2 (800 mg, 1.36 mmol) was dissolved in 10 mL dichloromethane under argon and diisopropylethylamine (710 μL, 4.08 mmol) and 2-cyanoethyl N,N-diisopropylchlorophosphoramidite (455 μL, 2.05 mmol) were added and the solution was stirred for 15 minutes when TLC indicated complete reaction. Water (4 mL) was added, the solution was stirred for 10 min. and partitioned between CH₂Cl₂ (50 mL) and aqueous NaHCO₃ (30 mL). The organic phase was washed with aqueous sodium chloride (2×30 mL) and the aqueous phases were back extracted with CH₂Cl₂ (30 mL). Evaporation of the organics left an oil which was flash purified twice on 40 g of silica gel (hexane: acetone: TEA, 68:30:2) to afford the product as a foam after coevaporation with dichloromethane. Dissolution in 2 mL of dichloromethane and precipitation in 80 mL cold (−70° C.) hexane afforded 718 mg (0.91 mmol, 67%) of the title product 22.2 as a white powder.

Rf (hexane:acetone:TFA 49:49:2): 0.37; HRMS calcd. for C₄₂H₅₄N₄O₉P₁ M+H]⁺: 789.36281, found: 789.3640; ³¹p-NMR δ (ppm, external ref.=H₃PO₄ capil.) 148.55, 149.01; ¹³C-NMR (CDCl₃) δ: 163.43 (C4), 150.78 (C2), 137.98 (C6), 117.2 (CN), 110.68 (C5), 98.22 (C1′), 86.13 (Ph₃C), 72.57 (C5′), 63.42 (d, J=17.5 Hz, C4′), 62.08 (C6′), 58.10 and 57.65 (2×d, J=18.6 Hz, POCH₂), 55.20 (2×CH₃O), 54.88 (1′-OCH₃), 53.60 (C2′), 43.11 (2×PNCH), 31.90 (C3′), 24.60 and 24.20 (4×CHCH₃), 20.20 (CH₂CN), 12.49 (5-CH₃)+aromatic signals.

### EXAMPLE 13

### 3′-O-methylation (2)

Methyl 4,6-O-benzylidene-3-O-methyl-2-(thymin-1-yl)-2-deoxy-D-altro-hexopyranoside (23.2).

An amount of 2.23 g (5.71 mmol) of 18.2 was coevaporated twice with anhydrous acetonitrile, dissolved in 70 ml dry THF and cooled on an ice bath. A 60% NaH dispersion in oil was added (685 mg, 17.1 mmol) and the mixture was stirred for 30 min. at 0° C., after which methyl iodide (1.8 ml, 29 mmol) was added. After stirring for 5 h the reaction remained incomplete and an additional amount of 1.2 ml of iodomethane was added and the mixture was stirred for 16 h more at 4° C. The reaction was quenched through addition of 20 ml of water and stirring for 20 min. after which the mixture was partitioned between 200 ml of ethyl acetate and 100 ml of 5% aqueous NaHCO₃. The organics were washed once more with NaHCO₃ and the aqueous phases were back extracted with 100 ml of EtOAc. Chromatographic purification on silica gel was tedious and following two column purifications, 790 mg (1.95 mmol, 34%) of the title product 23.2 was isolated as a foam, in addition to 166 mg (0.4 mmol, 7%) of the bis-methylated derivative (base and sugar methylated).

UV (MeOH) λmax 268 nm, λmin 234 nm; ESI-MS pos.: HRMS calcd. for C₂₀H₂₅N₂O₇ 405.16616; found 405.1693 [M+H]⁺, ¹H-NMR (CDCl₃): δ: 1.98 (s, 3H, 5-CH₃), 3.46 (s, 3H, 1′-OCH₃), 3.61 (s, 3H, 3′-OCH₃), 3.65-3.85 (m, 3H, H5′, H4′, H6′a), 4.354.52 (m, 2H, H3′, H6′e), 4.82 (s, 1H, H1′), 4.88 (d, 1H, J=1.5 Hz, H2′), 5.57 (s, 1H, PhCH, 7.32-7.50 (m, 5H, arom-H), 7.60 (d, J=0.7 Hz, 1H, H6), 9.66 (s, 1, NH); ¹³C-NMR (CDCl₃) δ: 163.83 (C4), 150.69 (C2), 136.85 (C6), 137.15, 129.20, 128.32, 126.26(Ph), 111.41 (C-5), 102.40 (PhCH), 98.88 (C1′), 76.36 (C3′), 76.03 (C4′), 69.17 (C6′), 59.49 (C2′), 58.52 (C5′), 55.88 and 55.72 (2×OCH₃), 12.75 (5-CH₃);

### EXAMPLE 14

### Benzylidene Cleavage (4)

Methyl 3-O-methyl-2-(thymin-1-yl)-2-deoxy-D-altro-hexopyranoside (4.2).

The benzylidene protected analogue 23.2 (609 mg, 1.51 mmol) was dissolved in 20 ml of methanol and the solution was purged with nitrogen for 5 min. after which was added 0.2 ml of acetic acid and 200 mg of Pd 10% on C. The mixture was hydrogenated for 15 h on a Parr apparatus, filtered, evaporated and coevaporated with toluene to remove the acid traces, yielding the title product 4.2 as a white powder (413 mg, 1.31 mmol, 86%).

UV (MeOH) λmax 268 nm, λmin 234 nm; ESI-MS pos.: HRMS calcd. for C₁₃H₂₀N₂O₇Na 339.1168; found 339.1192 [M+Na]⁺; ¹H-NMR (DMSO-d₆+D₂O, 65° C.): δ: 1.75 (s, 3H, 5-CH₃), 3.20 and 3.24 (2×s, 2×3H, 1′-OCH₃, 3′-OCH₃), 3.61 (dAB, 2H, J_{5,A}=5.7 Hz, J_{5,B}=5.1 Hz, J_{A,B}=12.0 Hz, H6′), 3.75 (brd, J=6Hz, 1H, H3′), 3.80 (q, J=5.0 Hz, 1H, H5′), 4.03 (t, =4.1 Hz, 1H, H4′), 4.27 (br diffuse, H2′), 4.86 (d, J=6.0 Hz, 1H, H1′), 7.37 (s, 1H, H6); ¹³C-NMR (DMSO-d6+D₂O) δ: 163.84 (C4), 151.23 (C2), 108.81 (C-5), 98.59 (C1′), 77.08 (C5′), 75.5 (broad, C3′), 63.31 (C4′), 60.73 (C6′), 56.22 and 54.88 (2×OCH₃), 11.91 (5-CH₃); C6 and C2′ missing, very faint broad peak around 138 ppm for C6.

### EXAMPLE 15

### 6′-O-protection (3)

Methyl 6-O-dimethoxytrityl-3-O-methyl-2-(thymin-1-yl)-2-deoxy-D-altro-hexopyranoside (24.2).

The 3′-O-methylated thymidine analogue 4.2 (370 mg, 1.17 mmol) was coevaporated with anhydrous pyridine and was subsequently dissolved in 25 mL of dry pyridine to which dimethoxytrityl chloride (440 mg, 1.3 mmol) was added. The mixture was stirred for 4 h at ambient temperature, quenched with 2 mL of methanol and neutralized with some aqueous sodium bicarbonate. The mixture was concentrated and partitioned twice between dichloromethane and aqueous sodium bicarbonate. The organic layer was purified on 40 g of silica gel with a methanol step gradient (0 to 2%) in dichloromethane containing 0.5% of pyridine, affording 656 mg (1.06 mmol, 90%) of the title compound 24.2 as a light yellow foam.

ESI-MS pos.: calcd. for C₃₄H₃₈N₂O₉Na 641.2; found 641.2 [M+Na]⁺; ¹H-NM (CDCl₃): δ: 1.86 (s, 3H, 5-CH₃), 3.38-3.48 (m, 8H, 1′-OCH₃, 3′-OCH₃, H6′), 3.79 (s, 6H, OMe DMTr), 3.85-4.08 (m, 2H, H5′, H4′), 4.97 (d, J=6.0 Hz, 1H, H1′), 6.83 (d, J=8.8 Hz, arom-H), 7.14-7.38 (m, 10H, H6, arom-H), 9.16 (s, 1H, NH); H2′ and H3′ missing. ¹³C-NMR (CDCl₃): δ: 163.82 (C4), 150.78 (C2), 110.8 (faint, C-5), 98.55 (C1′), 86.20 (Ph₃C),=77.08 (C5′),=75.5 (broad, C3′), 64.46 (C4′), 63.10 (C6′), 57.90 and 55.62 (2×OCH₃), 55.18 (2×OMe DMTr), 12.38 (5-CH₃); C6 and C2′, C3′ and C5′ missing.

### EXAMPLE 16

### Phosphytilation (3)

Methyl 6-O-dimethoxytrityl-3-O-methyl-2-(thymin-1-yl)-2-deoxy-4-O-(P-β-cyanoethyl-N,N-diisopropylaminophosphinyl)-D-altro-hexopyranoside (25.2).

The dimethoxytritylated derivative 24.2 (625 mg, 1.01 mmol) was dissolved in 6 mL dichloromethane under argon and diisopropylethylamine (530 μL, 3.03 mmol) and 2-cyanoethyl N,N-diisopropylchlorophosphoramidite (340 μL, 1.5 mmol) were added and the solution was stirred for 90 minutes when TLC indicated complete reaction. Water (2 mL) was added, the solution was stirred for 10 min. and partitioned between CH₂Cl₂ (50 mL) and aqueous NaHCO₃ (30 mL). The organic phase was washed with aqueous sodium chloride (2×30 mL) and the aqueous phases were back extracted with CH₂Cl₂ (30 mL). Evaporation of the organics left an oil which was flash purified twice on 40 g of silica gel (hexane: acetone: TEA, 64:34:2) to afford the product as a foam after coevaporation with dichloromethane. Dissolution in 2 mL of dichloromethane and precipitation in 100 mL cold (−70° C.) hexane afforded 713 mg (0.87 mmol, 86%) of the title product 25.2 as a white powder.

Rf (hexane: acetone: TEA 49:49:2): 0.47; ESI-MS pos.: HRMS calcd. for C₄₃H₅₆N₄O₁₀P₁ 819.3734; found 819.3745 [M+H]⁺; ³¹P NMR δ (pm, external ref.=H₃PO₄ capil.) 151.46 (very weak signal).

### EXAMPLE 17

### Synthesis of 1-O-methyl Hexitol Nucleoside Analogues (2)

Methyl 4,6-O-benzylidene-2-(adenin-9-yl)-2-deoxy-D-altro-hexopyranoside (18.5).

Adenine (6.08 g, 45 mmol) was suspended in 200 ml of anhydrous DMF to which was added 1.68 g of a 60% oil dispersion of sodium hydride (42 mmol) and the mixture was heated on an oil bath for 1 hour at 90° C. The methyl alloside epoxide 17 (see references [32] and f33]; 3.96 g, 15 mmol) was added and the mixture was heated over night at 120° C., after which the reaction was cooled, quenched with sodium bicarbonate and concentrated. The residue was partitioned between 200 ml of ethyl acetate and 200 ml of 5% aqueous sodium bicarbonate, the aqueous phase was extracted once more with ethyl acetate and the organics were washed twice with brine. Following adsorption on 30 g of silica gel, the organic residue was purified by chromato-graphy on in total 120 g of silica gel (0-2% MeOH/dichloromethane) affording 4.59 g (11.5 mmol, 77%) of the title compound 18.5 as a foam.

¹H-NMR (CDCl₃): δ: 3.54 (s, 3H, OCH₃), 3.74 (dd, 1H, J=3.0 and 9.8 Hz, H4′), 3.86 (t, 1H, J=10.3 Hz, H6′a), 4.35 (brs, 1H, H3′), 4.45 (dd, J=5 and 10.5 Hz, H6′e), 4.59 (m, 1H, H5′), 5.06 (s, 1H, H1′), 5.15 (d, 1H, J=2.4 Hz, H2′), 5.20 (brs, 1H, OH), 5.55 (s, 1H, PhCH), 6.07 (s, 2H, NH₂), 7.28-7.42 (m, 5H, arom-H), 8.18 (s, 1H, H8), 8.30 (s, 1H, H2); ¹³C-NMR (CDCl₃) δ: 155.68 (C6), 153.45 (C-2), 149.79 (C4), 138.45 (C8), 136.92 (Ci), 129.24 (Cp), 128.22 (Cm), 126.15 (Co), 118.92 (C5), 102.33 (PhCH), 99.68 (C1′), 75.91 (C4′), 69.16 (C6′), 67.06 (C3′), 58.55 (C5′), 57.28 (C2′), 56.07 (OCH₃); ESI-MS pos.: 400 [M+H]⁺, 368 [M-CH₃OH]⁺, 294 [M-BnOH]⁺, 262 [M-BnOH—CH₃OH]⁺, 136 [BH₂]⁺; HRMS calcd. for C₁₉H₂₂N₅O₅ 400.1620; found 400.1620 [M+H]⁺.

### EXAMPLE 18

### 3′-O-methylation (3)

Methyl 4,6-O-benylidene-3-O-methyl-2-(adenin-9-yl)-2-deoxy-D-altro-hexopyranoside (23.5).

An amount of 2.0 g (5 mmol) of 18.5 was dissolved under argon in 80 ml dry DMF and cooled on an ice bath. A 60% NaH dispersion in oil was added (280 mg, 7 mmol) and the mixture was stirred for 45 min. at 0° C., after which methyl iodide (0.5 ml 8 mmol) dissolved in 50 ml of DMF was added over 2 hours. After stirring for 3 h at 0° C., the reaction was left for 1 h more at room temperature and was quenched through addition of 10 ml of water and stirred for 10 min. more. Following concentration, the mixture was partitioned between 2×200 ml of ethyl acetate and 2×150 ml of 5% aqueous NaHCO₃ and 1×100 ml of brine. Chromatographic purification on silica gel (CH₂Cl₂ to CH₂Cl₂-MeOH 96:4) afforded several products methylated at the sugar ring among which the title product 23.5 (1055 mg, 2.55 mmol, 51%) in addition to the N-bismethylated (155 mg, 0.35 mmol, 7%; HRMS calcd. for C₂₂H₂₈N₅O₅ 442.2090; found 442.2098 [M+H]⁺) and N-monomethylated (385 mg, 0.9 mmol, 18%; HRMS calcd. for C₂₁H₂₆N₅O₅ 428.1934; found 428.1930 [+H]⁺) derivatives. In addition 160 mg (0.4 mmol, 8%) of the starting nucleoside was recovered.

ESI-MS pos.: 414 [M+H]⁺, 382 [M-CH₃OH)⁺, 308 [M-BnOH]⁺, 276 [M-BnOH—CH₃OH]⁺, 244 [M-BnOH−2×CH₃OH]⁺, 136 [BH₂]⁺; HRMS calcd. for C₂₀H₂₄N₅O₅ 414.1777; found 414.1779 [M+H]⁺; ¹H-NMR (CDCl₃): δ: 3.51 (s, 3H, OCH₃), 3.71 (s, 3H, OCH₃), 3.76-3.92 (m, 3H, H4′, H6′a, H6′e), 4.38-4.62 (m, 2H, H3′, H5′), 5.07 (s, 1H, H1′), 5.15 (d, 1H, J=2.2 Hz, H2′), 5.49 (s, 1H, PhCH), 6.29 (s, 2H, NH₂), 7.30-7.46 (m, 5H, arom-H), 8.22 (s, 1H, H8), 8.40 (s, 1H, H2); ¹³C-NMR (CDCl₃) δ: 155.84 (C6), 153.57 (C-2), 150.02 (C4), 138.54 (C8), 137.15 (Ci), 129.16 (Cp), 128.29 (Cm), 126.19 (Co), 119.09 (C5), 102.46 (PhCH), 99.46 (C1′), 76.18 (C4′ and C3′), 69.23 (C6′), 60.00 (C2′), 58.75 (C5′), 56.00 (1′-OCH₃), 54.99 (3′-OCH₃).

### EXAMPLE 19

### Benzylidene Cleavage (5)

Methyl 3-O-methyl-2-(adenin-9-yl)-2-deoxy-D-altro-hexopyranoside (4.5)

The methylated adenosine analogue 23.5 (2.11 g, 5.1 mmol) was dissolved in 70 ml of dioxane, purged with nitrogen for 10 min. and 500 mg Pd on carbon and 3 g of ammonium acetate were added. The mixture was gently refluxed for 48 h with intermittent addition of fresh ammonium acetate. Filtration, adsorption on silica gel and evaporation was followed by column purification on silica gel (gradient from CH₂Cl₂ to CH₂Cl₂—MeOH 9:1) affording 750 mg of the deprotected compound 4.5 (2.3 mmol, 45%), while another 850 mg (40%) of the starting product was still recovered.

ESI-MS pos.: 326 [M+H]⁺, 294 [M-CH₃OH]⁺, 262 [M-2×CH₃OH]⁺, 136 [BH₂]⁺; HRMS calcd. for C₁₃H₂₀N₅O₅ 326.1464; found 326.1465 [M+H]⁺; ¹H-NMR (D)MSO-d6) δ: 3.09 and 3.21 (2×s, 2×3H, 1′-OCH₃, 3′-OCH₃), 3.70 (tAB, 2H, J_{5,A}=6.1 Hz, J_{5,B}=5.5 Hz, J_{A,B}=12.5 Hz,. H6′), 3.89 (dt, J=5.7 and 4.2 Hz, 1H, H5′), 4.09 (q, 1H, J=4.0 Hz, H4′), 4.12 (dd, 1H, J=3.5 and 9.7 Hz, H3′), 4.54 (dd, 1H, J_{2,1}=6.8 Hz, J_{2,3}−9.8 Hz, H2′), 4.91 (t, J=5.6 Hz, 1H, 6′-OH1), 4.96 (d, J=4.9 Hz, 1H, 4′-OH), 5.13 (d, J=6.3 Hz, 1H, H1′), 7.18 (s, 2H, NH₂), 8.13 (s, 1H, H2), 8.20 (s, 1H, H8); ¹³C-NMR (DMSO-d6+D₂O) δ: 156.06 (C6), 152.30 (C-2), 149.85 (C4), 140.97 (C8), 119.03 (C5), 98.49 (C1′), 77.08 (C5′), 76.10 (C3′), 63.10 (C4′), 60.42 (C6′), 56.31 (C2′), 56.26 (1′-OCH₃), 54.99 (3′-OCH₃).

### EXAMPLE 20

### One Pot 6′-O-Protection and Base Protection

Methyl 6-O-dimethoxytrityl-3-O-methyl-2-(N⁶-benzoyl-adenin-9-yl)-2-deoxy-D-altro-hexopyranoside (24.6)

According to the procedure of reference [42], an amount of 880 mg (2.7 mmol) of 4.5 was coevaporated twice with pyridine and dissolved in 50 ml of anhydrous pyridine. The nucleoside analogue was reacted accordingly with 4,4′-dimethoxytrityl chloride, followed by reaction with benzoyl chloride, and the reaction was worked-up as described. Chromatographic purification yielded 1460 mg (2.0 mmol, 744%) of the title compound as a slightly yellow foam.

ESI-MS pos.: HRMS calcd. for C₄₁H₄₂N₅O₈ 732.3033; found 732.3041 [M+H]⁺; calcd. for C₄₁H₄₁N₅O₈Na 754.2853; found 754.2855 [M+Na]⁺;

### EXAMPLE 21

### Phosphytilation (4)

Methyl 6-O-dimethoxytrityl-3-O-methyl-2-N⁶-benzoyl-adenin-9-yl)-2-deoxy-4-O-(P-βcyanoethyl-N,N-diisopropylaminophosphinyl)-D-altro-hexopyranoside (25.6).

The dimethoxytritylated derivative 24.6 (795 mg, 1.08 mmol) was dissolved in 6 mL dichloromethane under argon and diisopropylethylamine (570 μL, 3.26 mmol) and 2-cyanoethyl N,N-diisopropylchlorophosphoramidite (390 μL, 1.73 mmol) were added and the solution was stirred for 45 minutes when TLC indicated complete reaction. Water (2 mL) was added, and the reaction was worked-up as for the synthesis of 25.2 (example 16). Precipitation in 100 mL cold (−70° C.) hexane afforded 880 mg (0.94 mmol, 86%) of the title product 25.6 as a white powder.

Rf (hexane: acetone: TEA 49:49:2): 0.51; ESI-MS pos.: HRMS calcd. for C₅₀H₅₉N₇O₉P₁ 932.4112; found 932.4128 [M+H]⁺; calcd. for C₅₀H₅₈N₇O₉P₁Na₁ 954.3931; found 954.3920 [M+H]⁺; ³¹P NMR δ (ppm, external ref.=H₃PO₄ capil.) 151.81 and 150.92.

### EXAMPLE 22

### Synthesis of New Altritol Nucleoside Analogues (1)

1,5-Anhydro-4,6-O-benzylidene-2-(thymin-1-yl)-2-deoxy-D-altro-hexitol (7.2)

Thymine (3.78 g, 20 mmol) was suspended in 200 ml of anhydrous DMF to which was added under argon 1.08 g of a 60% oil dispersion of sodium hydride (27 mmol) and the mixture was heated on an oil bath for 1 hour at 90° C. The allitol epoxide 6 (see reference [14]; 3.51 g, 15 mmol) was added and the mixture was heated for 18 h at 120° C., after which the reaction was cooled, quenched with sodium bicarbonate and concentrated. The residue was partitioned between 400 ml of ethyl acetate and 400 ml of 5% aqueous sodium bicarbonate, and the organics were washed twice with brine. The aqueous phases were back extracted twice with ethyl acetate, and the organic residue was purified on 200 g of silica gel (slow gradient of MeOH in CH₂Cl₂ 0-2%) affording 3.07 g (8.52 mmol, 57%) of the title compound 7.2 as a foam.

ESI-MS pos. HRMS calcd. for C₁₈H₂₁N₂O₆ 361.1400; found 361.1398 [M+H]⁺; ¹H-NMR (DMSO-d₆): δ: 1.96 (s, 3H, 5-CH₃), 3.64 (dd, 1H, J-2.8 and 9.7 Hz, H4′), 3.68 (brs, 1H, OH), 3.72 (t, 1H, J=10.5 Hz, H6′a), 3.99 (d, 1H, J=13.9 Hz, H1′a), 4.12 (dt, 1H, 3=4.9 and 10.0 Hz, H5′), 4.30(m, 1H, H3′), 4.36-4.42 (m, 2H, H6′e, H1′e), 4.48 (t, 1H, J=2.8 Hz, H2′), 5.61 (s, 1H, PhCH), 7.32-7.47 (m, 5H, arom-H), 7.83 (s, 1H, H6), 9.59 (s, 1H, NH); ¹³C-NMR (CDCl₃) δ: 163.73 (C-4), 151.12 (C-2), 137.70 (C-6), 137.00, 129.20, 128.21, 126.17 (Ph), 111.42 (C-5), 102.25 (PhCH), 98.59 (C-1′), 76.75 (C4′), 68.99 (C-6′), 66.32. (C-5′), 66.11 (C-3′), 64.20 (C1′), 56.69 (C-2′), 12.77 (5-CH₃).

### EXAMPLE 23

### Synthesis of New Altritol Nucleoside Analogues (2)

1,5-Anhydro-4,6-O-benzylidene-2-(triazol-1-yl-3-methylcarboxylate)-2-deoxy-D-altro-hexitol (7.7).

1,2,4-Triazole-3-methylcarboxylate (1.27 g, 10 mmol; Alkemie, Lokeren, Belgium) was suspended in 50 ml of anhydrous DMF to which was added under argon 380 mg of a 60% oil dispersion of sodium hydride (9.5 mmol) and the mixture was heated on an oil bath for 30 min. at 90° C. The allitol epoxide (see reference [14]) 6 (703 mg, 3 mmol) was added and the mixture was heated for 15 h at 90° C., after which the reaction was cooled, quenched with sodium bicarbonate and concentrated. The residue was partitioned between 200 ml of ethyl acetate and 200 ml of 5% aqueous sodium bicarbonate, and the organics were washed twice with brine. Purification of the organic residue on 70 g of silica gel (slow gradient of MeOH in CH₂Cl₂ 0-2%) afforded 560 mg (1.55 mmol, 52%) of the title compound 7.7 as a foam.

UV (MeOH) λmax 206, 220 (sh) nm; ¹H-NMR (CDCl₃): δ: 3.52 (dd, 1H, J=2.4 and 9.8 Hz, H4′), 3.71 (t, 1H, J=10.2 Hz, H6′a), 4.02 (s, 3H, COOCH₃), 4.08 (dt, 1H, J=4.9 and 10.0 Hz, H5′), 4.26 (d, 1H, J=13.2 Hz, H1′a), 4.37 (dd, 1H, J=4.9 and 10.2 Hz, H6′e), 4.42 (dd, 1H, J=2.4 and 13.6 Hz, H1′e), 4.62 (m, 2H, H2′, H3′), 5.53 (s, 1H, PhCH), 7.33-7.44 (m, 5H, arom-H), 8.54 (s, 1H, H5); ¹³C-NMR (CDCl₃) δ: 159.96 (CO), 154.86 (C3), 144.40 (CS), 136.75 (Ci), 129.33 (Cp), 128.31 (Co), 126.06 (Cm), 102.16 (PhCH), 76.18 (C4′), 68.78 (C6′), 67.31 (C5′), 66.92 (C3′), 64.32 (C1′), 61.13 (C2′), 52.83 (OCH₃); ESI-MS pos.: HRMS calcd. for C₁₇H₂₀N₃O₆ 362.1352; found 362.1356 [M+H]⁺.

### EXAMPLE 24

### Benzylidene Cleavage (6)

1,5-Anhydro-2-(triazol-1-yl-3-methylcarboxylate)-2deoxy-D-altro-hexitol (13.7)

The triazole methylcarboxylate analogue 7.7 (400 mg, 1.1 mmol) was dissolved in 20 ml of methanol and 1 ml of TFA was added. The mixture was stirred for 3 h after which the mixture was evaporated and coevaporated with dioxane. The title product 13.7 partially crystallized from a methanol—toluene mixture affording 144 mg (0.52 mmol, 48%) of the title compound.

¹H-NMR (DMSO): δ: 3.30 (s, 3H, OCH₃), 3.39-3.44 (m, 1H, H4′), 3.52 (dD, J_{5′,6′B}=4.9 and J_{6′,6′}=11.2 Hz, 1H, H6′B), 3.57-3.64 (m, 2H, H5′, H6′A), 4.01 (dD, 1H, J=3.4 and 12.7 Hz, H1′ₐ), 4.12 (dD, 1H, J=3.0 and 12.7 Hz, H1′ₐ), 4.14 (dd, 1H, H3′), 4.50 (Dt, 1H, H2′), 4.56 (t, 1H, J=5.6 Hz, 6′-OH), 4.80 (d, 1H, 3=6.3 Hz, 3′-OH), 5.40 (d, 1H, J=4.4 Hz, 4′-H), 8.76 (s, 1H, H5); ¹³C-NMR (CDCl₃) δ: 159.93 (CO), 153.43 (C3), 145.55 (CS), 77.84 (C5′), 67.87 (C3′), 64A9 (C4′), 62.79 (C1′), 60.97 (C2′), 60.39 (C6′), 52.14 (OCH₃); ESI-MS pos.: HRMS calcd. for C₁₀H₁₆N₃O₆ 274.1039; found 274.1048 [M+H]⁺; HRMS calcd. for C₁₀H₁₅N₃O₆Na 296.0859; found 296.0859 [M+Na]⁺.

### EXAMPLE 25

### Ester to Amide Conversion

1,5Anhydro-4,6-O-benzylidene-2-(triazol-1-yl-3-carboxamide)-2-deoxy-D-altro-hexitol (7.8).

The triazole ester 7.7 (530 mg, 1.47 mmol) was dissolved in 30 ml of a 2 M solution of ammonia in methanol and stirred overnight at room temperature, after which the mixture was evaporated and the residue was adsorbed on silica gel. Chromatographic purification (gradient of MeOH in CH₂Cl₂ 0-5%) yielded 380 mg of the amide 7.8 (1.1 mmol, 75%) as a foam.

UV (MeOH) λmax 214 nm; ¹H-NMR (CDCl₃+CD₃OD): δ: 3.55 (dd, 1H, J=2.0 and 9.8 Hz, H4′), 3.70 (t, 1H, J=10.5 Hz, H6′a), 4.08 (dt, 1H, J=4.9 and 10.0 Hz, H5′), 4.29 (dd, 1H, J=5.3 and 10.2 Hz, H6′e), 4.30 (d, 1H, J=13.7 Hz, H1′a), 4.39 (dd, 1H, 3=1.7 and 13.5 Hz, H1′e), 4.53 (m, 2H, H2′, H3′), 5.52 (s, 1H, PhCH), 7.33-7.44 (m, 5H, arom-H), 8.59 (s, 1H, H5); ¹³C-NMR (CDCl₃) δ: 161.22 (CO), 155.89 (C3), 143.94 (CS), 136.75 (Ci), 128.35 (Cp), 127.40 (Co), 125.61 (Cm), 101.61 (PhCH), 75.85 (C4′), 68.16 (C6′), 66.36 (C5′), 65.82 (C3′), 63.47 (C1′), 61.52 (C2′); ESI-MS pos.: HMS calcd. for C₁₆H₁₈N₄O₅ 347.1355; found 347.1328 [M+H]⁺.

### EXAMPLE 26

### Benzylidene Cleavage (7)

1,5-Anhydro-2-(triazol-1-yl-3-carboxamide)-2-deoxy-D-altro-hexitol (13.8).

The triazole carboxamide analogue 7.8 (380 mg, 1.1 mmol) was dissolved in 20 ml of methanol and 1 ml of TFA was added. The mixture was stirred for 4 h after which a precipitate was formed. After evaporation and coevaporation with dioxane the mixture was crystallized from boiling ethanol affording 223 mg (0.86 mmol, 78%) of the title compound 13.8.

UV (MeOH) λmax 214 nm; ¹H-NMR (DMSO): δ: 3.38 (dd, 1H, J_{3′,4′}=3.2 and J_{4′,5′}=7.8 Hz, H4′), 3.52 (dD, J_{5′,6′B}=5.0 and J_{6′,6′}=11.3 Hz, 1H, H6′B), 3.60 (ddd, J=2.9, 5.0 and 7.9 Hz, 1H, H5′), 3.63 (dD, J_{5′,6′A}=2.9 and J_{6′,6′}=11.3 Hz, 1H, H6′A), 4.01 (dD, 1H, J=3.2 and 12.5 Hz, H1′), 4.12 (dD, 1H, J=3.0 and 12.5 Hz, H1′), 4.14 (dd, 1H, J=3.2 and 4.9 Hz, H3′), 4.45 (Dt, 1H, J_{2′,3′}=5.1 and J_{2′,1′}3.2 Hz, H2′), 4.55 (t, 1H, 6′-OH), 4.78 (d, 1H, J=5.8 Hz, 4′-OH), 5.40 (d, 1H, J=4.4 Hz, 3′-OH), 7.54 (s, 1H) and 7.75 (s, 1H) (NH₂), 8.67 (s, 1H, H5); ¹³C-NMR (CDCl₃) δ: 160.55 (CO), 156.53 (C3), 144.70 (C5), 77.73 (C5′), 67.67 (C3′), 64.20 (C4′), 62.62 (C1′), 60.59 (C2′), 60.21 (C6′); ESI-MS pos.: HRMS calcd. for C₉H₁₅N₄O₅ 259.1042; found 259.1048 M+H]⁺.

### EXAMPLE 27

### 3′-O-methylation (4)

1,5-Anhydro-4,6O-benzylidene-3-O-methyl-2-triazol-1-yl-3-methylcarboxylate)-2-deoxy-D-altro-hexitol (8.7)

An amount of 900 mg (2.5 mmol) of 7.7 was dissolved under argon in 20 ml dry DMF and cooled on an ice bath. A 60% NaH dispersion in oil was added (120 mg, 3 mmol) and the mixture was stirred for 45 min. at 0° C., after which methyl iodide (0.25 ml, 4 mmol) dissolved in 10 ml of DMF was added over 30 min. After stirring for 3 h at room temperature, the reaction was quenched with 3 ml of water and stirred for 10 min. more. Following concentration, the mixture was partitioned between 2×70 ml of ethyl acetate and 2×50 ml of 5% aqueous NaHCO₃ and 1×50 ml of brine. Chromatographic purification on silica gel (CH₂Cl₂ to CH₂Cl₂—MeOH 98:2) afforded 647 mg (1.72 mmol, 70%) of the triazole derivative 8.7 methylated on the hexitol ring.

ESI-MS pos. calcd. for C₁₈H₂₂N₃O₆ 376.1509; found 376.1496 [M+H]⁺; ¹H-NMR (CDCl₃): δ: 3.44 (dd, 1H, J=2.7 and 9.5 Hz, H4′), 3.65 (t, 1H, J=10.5 Hz, H6′A), 3.66 (s, 3H, 3′-OCH₃), 4.03 (s, 3H, COOCH₃), 4.11 (dt, 1H, J=5.1 and 9.9 Hz, H5′), 4.23 (t, 1H, J=5.9 Hz, H3′), 4.31 (d, 1H, J=13.2 Hz, H1′a), 4.34 (dd, 1H, J=5.4 and 10.2 Hz, H6′e), 4.38 (dd, 1H, J=2.4 and 13.2 Hz, H1′e), 4.61 (t, 1H, J=2.4 Hz, H2′), 5.40 (s, 1H, PhCH), 7.33-7.45 (m, 5H, arom-H), 8.57 (s, 1H, H5); ¹³C-NMR (CDCl₃) δ: 160.02 (CO), 155.00 (C3), 144.40 (C5), 137.20 (Ci), 129.10 (Cp), 128.24 (Co), 126.09 (Cm), 102.42 (PhCH), 75.54 (C4′), 68.93 (C6′), 67.55 (C5′), 64.16 (C3′), 60.76 and 60.23 (C1′, C2′), 52.79 (2×OCH₃).

### EXAMPLE 28

### Ester to Amide Conversion and Benzylidene Cleavage

1,5-Anhydro-3-O-methyl-2-(triazol-1-yl-3-carboxamide)-2-deoxy-D-altro-hexitol (1.8)

The triazole ester derivative 8.7 (408 mg, 1.08 mmol) was dissolved in 25 ml of a 2 M solution of ammonia in methanol and stirred overnight at room temperature, after which the mixture was evaporated and the residue was adsorbed on silica gel. Purification on silica gel (gradient of MeOH in CH₂Cl₂ 0-5%) yielded 350 mg (0.97 mmol, 90%) of 8.8 as a foam.

ESI-MS pos. calcd. for C₁₇H₂₁N₄O₅ 3601.1512; found 361.76.1534 [M+H]⁺.

The foam was dissolved in 25 ml of methanol, 1.25 ml of TFA were added and the mixture was stirred for 4 h at room temperature. Evaporation left an oil which was crystallized from methanol—diisopropylether, affording the title compound 1.8 (188 mg, 0.69 mmol, 71%).

¹H-NMR (DMSO-d₆): δ: 3.42 (s, 3H, OCH₃), 3.52 (dD, J_{5′,6′B}=4.7 and J_{6′,6′}=11.0 Hz, 1H, H6′B), 3.54-3.60 (m, 2H, H4′, H5′), 3.62 (dD, J_{5′,6′A}=2.9 and J_{6′,6′}=11.3 Hz, 1H, H6′A), 3.87 (dd, 1H, J=2.9 and 4.9 Hz, H3′), 3.94 (dD, 1H, J=3.9 and 12.7 Hz, H1′ₐ), 4.15 (dD, 1H, J=3.6 and 12.5 Hz, H1′ₑ), 4.66 (Dt, 1H, J_{2′,3′}=4.9 and J_{2′,1′}=3.5 Hz, H2′), 4.6-5.0 (br, 2×OH), 7.56 (s, 1H) and 7.77 (s, 1H) (NH₂), 8.70 (s, 1H, H5); ¹³C-NMR (CDCl₃) δ: 160.53 (CO), 156.65 (C3), 144.93 (C5), 78.38, 77.91 (C3′, C5′), 63.38, 63.12 (C4′, C1′), 60.21, 58.08, 57.80 (C2′, C6′, OMe); ESI-MS pos.: HRMS calcd. for C₁₀H₁₇N₄O₅ 273.1199; found 273.1189 [M+H]⁺.

### EXAMPLE 29

### Synthesis of 1-O-methyl Hexitol Nucleoside Analogues (3)

Methyl 4,6-O-benzylidene-2(triazol-1-yl-3-methylcarboxylate)-2-deoxy-altro-hexopyranoside (18.7).

1,2,4-Triazole-3-methylcarboxylate (5.72 g, 45 mmol; Alkemie, Lokeren, Belgium) was suspended in 100 ml of anhydrous DMF to which was added under argon 1.72 g of a 60% oil dispersion of sodium hydride (43 mmol) and the mixture was heated on an oil bath for 1 hour at 90° C. The methyl alloside epoxide 17 (see references [32] and [33]; 3.52 g, 15 mmol) was added and the mixture was heated for 48 h at 90° C., when TLC analysis indicated a multitude of products. The reaction was cooled, quenched with sodium bicarbonate and concentrated. The residue was partitioned between 200 ml of ethyl acetate and 200 ml of 5% aqueous sodium bicarbonate, the aqueous phase was extracted two times more, and the organics were washed twice with brine. Purification of the organic residue on 70 g of silica gel (slow gradient of MeOH in CH₂Cl₂ 0-2%) afforded 1220 mg (3.12 mmol, 21%) of the title compound 18.7 as a foam, which crystallized from acetone—diisopropyl ether. In addition 800 mg of the epoxide (3.0 mmol, 20%) was recovered, and 291 mg (0.87 mmol, 6%) of the decarboxylated triazolide derivative 18.9 (B=1,2,4-triazol-1-yl) was isolated as a foam

18.7: ¹H-NMR (CDCl₃): δ: 3.52 (s, 3H, 1′-OCH₃), 3.87 (t, J=11.7 Hz, 1H, H6′A), 3.89 (dd, J=3.4 and 9.3 Hz, 1H, H4′), 4.01 (s, 3H, COOCH₃), 4.39-4.45 (m, 3H, H3′, H5′, H6′e), 4.87 (d, 1H, J=2.4 Hz, H2′), 5.05 (s, 1H, H1′), 5.62 (s, 1H, PhCH), 7.34-7.46 (m, 5H, arom-H), 8.40 (s, 1H, H5); ¹³C-NMR (CDCl₃) δ: 159.81 (CO), 155.11 (C3), 144.20 (C5), 136.78 (Ci), 129.31 (Cp), 128.29 (Cm), 126.15 (Co), 102.25 (PhCH), 98.95 (C1′), 75.26 (C4′), 68.92 (C6′), 67.75 (C3′), 62.88 (C2′), 58.31 (C5′), 56.03 (1′-OCH₃), 52.82 (COOCH₃); ESI-MS pos. HRMS calcd. for C₁₈H₂₂N₃O₇ 392.1458; found 392.1465 [M+H]⁺.

18.9: ¹H-NMR (CDCl₃): δ: 3.49 (s, 3H, 1′-OCH₃), 3.87 (t, J=12.0 Hz, 1H, H6′A), 4.03 (dd, J=3.2 and 9.1 Hz, 1H, H4′), 4.31 (brs, 1H, H3′), 4.37-4.44 (m, 2H, H5′, H6′e), 4.75 (d, 1H, J=2.4 Hz, H2′), 4.93 (s, 1H, H1′), 5.63 (s, 1H, PhCH), 7.34-7.46 (m, 5H, arom-H), 7.98 (s, 1H, H3), 8.22 (s, 1H, H5); ¹³C-NMR (CDCl₃) δ: 152.13 (C3), 142.97 (C5), 136.96 (Ci), 129.26 (Cp), 128.26 (Cm), 126.17 (Co), 102.19 (PhCH), 99.44 (C1′), 75.51 (C4′), 69.02 (C6′), 68.04 (C3′), 62.00 (C2′), 58.12 (C5′), 55.86 (1′-OCH₃); ESI-MS pos. HRMS calcd. for C₁₆H₂₀N₃O₅ 334.1403; found 334.1418 [M+H]⁺.

### EXAMPLE 30

### Ester to Amide Conversion (2)

Methyl 4,6-O-benzylidene-2-(triazol-1-yl-3-carb xamide)-2-deoxy-D-altro-hexopyranoside 18.8.

The methyl carboxylate 18.7 (500 mg, 1.28 mmol) was dissolved in 20 ml of a 2 M solution of ammonia in methanol and stirred overnight at room temperature, after which the mixture was evaporated and the residue was adsorbed on silica gel. Flash purification on silica gel (gradient of MeOH in CH₂Cl₂ 0-7%) yielded 450 mg (1.19 mmol, 93%) of 18.8 as a foam.

¹H-NMR (DMSO-d₆) δ: 3.35 (s, 3H, 1′-OCH₃), 3.82 (t, J=9.7 Hz, 1H, H6′A), 4.01 (dd, J=3.2 and 9.5 Hz, 1H, H4′), 4.17 (t, 1H, J=2.8 Hz, H3′), 4.20 (dd, 1H, J=5.3 and 9.9 Hz, H5′), 4.25 (dd, 1H, J=5.1 and 9.5 Hz, H6′e), 4.73 (d, 1H, J=2.5 Hz, H2′), 5.00 (s, 1H, H1′), 5.50 (d, 1H, 3′-OH), 5.73 (s, 1H, PhCH) 7.34-7.46 (m, 5H, arom-H), 8.74 (s, 1H, H5); ¹³C-NMR (DMSO-d6) δ: 160.38 (CO), 156.87 (C3), 145.11 (C5), 137.79 (Ci), 128.79 (Cp), 127.92 (Cm), 126.39 (Co), 100.95 (PhCH), 98.74 (C1′), 75.02 (C4′), 68.17 (C6′), 66.47 (C3′), 63.16 (C2′), 57.53 (C5′), 54.86 (1′-OCH₃); ESI-MS pos. HRMS calcd. for C₁₇H₂₁N₄O₆ 377.1461; found 377.1450 [M+H]⁺.

### EXAMPLE 31

### Benzylidene Cleavage (8)

Methyl 2-triazol-1-yl-3-carboxamide)-2deoxy-D-altro-hexopyranoside 3.8.

An amount of 504 mg (1.34 mmol) of the benzylidene protected compound 18.8 was dissolved in 25 mL of methanol, and 0.25 mL of acetic acid was added. The solution was degassed by bubbling nitrogen for 10 min. after which 250 mg of 10% Pd on carbon was added and the mixture was hydrogenated overnight on a Parr apparatus at 45 psi. The mixture was filtered, the filter was washed with hot ethanol and the volatiles were removed in vacuo. The residue was adsorbed on 4 g of silica gel and purified by column chromatography on 20 g of silica gel (gradient of MeOH in CH₂Cl₂ 0-15%) yielding 302 mg (1.05 mmol, 78%) of the title compound 3.8 as a foam,

¹H-NMR (DMSO-d₆): δ: 3.24 (s, 3H, 1′-OCH₃), 3.62 (m, 2H, H6′), 3.80-3.86 (m, 2H, H5′, H4′), 4.02-4.08 (m, 1H, H3′), 4.36 (dd, 1H, J_{2′,1′}=6.8 Hz, J_{2′,3′}=10.3 Hz, H2′), 4.88-4.96 (m, 2H, 3′-OH, 6′-O), 4.97 (d, 3=6.8 Hz, 1H; H1′), 5.04 (d, J=3 Hz, 1H, 4′-OH), 7.53 (s, 1H) and 7.73 (s, 1H) (NH₂), 8.65 (s, 1H, H5); ¹³C-NMR (DMSO-d₆) δ: 160.67 (CO), 157.11 (C3), 146.41 (C5), 99.10 (C1′), 77.41 (C5′), 67.62 (C3′), 67.07 (C4′), 62.52 (C2′), 60.97 (C6′), 55.13 (1′-OCH₃); ESI-MS pos. RMS calcd. for C₁₀H₁₇N₄O₆ 289.1148; found 289.1136 [M+H]⁺.

### EXAMPLE 32

### 3′-O-methylation (4) and Ester to Amide Conversion (3)

Methyl 4,6-O-benzylidene-3-O-methyl-2-(triazol-1-yl-3-carboxamide)-2-deoxy-D-altro-hexopyranoside (23.8)

The methyl carboxylate 18.7 (626 mg, 1.6 mmol) was dissolved under argon in 10 ml dry DMF and cooled on an ice bath. A 60% NaH dispersion in oil was added (84 mg, 1.9 mmol) and the mixture was stirred for 45 min. at 0° C., after which methyl iodide (0.16 ml, 2.6 mmol) dissolved in 10 ml of DMW was added over 30 min. After stirring for 2 h at room temperature, the reaction was quenched with 3 ml of water and stirred for 10 min. more. Following concentration, the mixture was partitioned between 2×50 ml of ethyl acetate and 2×50 ml of 5% aqueous NaHCO₃ and the organics were washed with 50 ml of brine. Chromatographic purification on silica gel (CH₂Cl₂ to CH₂Cl₂— MeOH 98:2) afforded 276 mg (0.68 mmol, 42%) of 3′-O-methylated derivative 23.7.

ESI-MS pos. HRMS calcd. for C₁₉H_{2,4}N₃O₇ 406.1614; found 406.1612 [M+H]⁺;

ESI-MS pos. HRMS calcd. for C₁₉H₂₃N₃O₇Na 428.1434; found 428.1429 [M+Na]⁺.

The methyl carboxylate 23.7 (276 mg, 0.68 mmol) was dissolved in 20 ml of a 2 M solution of ammonia in methanol to which was added 10 ml of dioxane, and the mixture was stirred overnight at room temperature, after which the mixture was evaporated and the residue was adsorbed on silica gel. Flash purification on silica gel (gradient of MeOH in CH₂Cl₂ 0-7%) yielded 228 mg (0.58 mmol, 86%) of the amide 23.8 as a foam

¹H-NMR (DMSO-d₆) δ: 3.35 (s, 3H, OCH₃), 3.47 (s, 3H, OCH₃), 3.81 (t, J=10.0 Hz, 1H, H6′A), 3.96 (t, 1H, J=2.4 Hz, B3′), 4.19 (dT, 1H, J=5.2 and 10.0 Hz, H5′), 4.25 (dD, J=3.4 and 9.7 Hz, 1H, H4′), 4.27 (dD, 1H, J=5.4 and 10.3 Hz, H6′e), 4.95 (d, 1H, J=2.0 Hz, H2′), 5.00 (s, 1H, H1′), 5.71 (s, 1H, PhCH) 7.35-7.44 (m, 5H, arom-H1), 7.63 (s, 1H) and 7.84 (s, 1H) (NH₂), 8.79 (s, 1H, H5); ¹³C-NMR (DMSO-d₆) δ: 160.38 (CO), 156.89 (C3), 145.53 (C5), 137.80 (Ci), 128.86 (Cp), 128.09 (Cm), 126.19 (Co), 101.00 (PhCH), 98.63 (C1′), 75.94 (C3′), 75.16 (C4′), 68.19 (C6′), 59.55 (C2′), 58.69 (3′-OMe), 58.09 (C5′), 54.94 (1′-OMe); ESI-MS pos. HRMS calcd. for C₁₈H₂₂N₄O₆Na 413.1437; found 413.1445 [M+Na]⁺.

### EXAMPLE 33

### Benzylidene Cleavage (9)

Methyl 3-O-methyl-2-(triazol-1-yl-3-carboxamide)-2-deoxy-D altro-hexopyranoside (4.8)

An amount of 218 mg (0.56 mmol) of the benzylidene protected compound 23.8 was treated as for the synthesis of 3.8 (example 29) affording 151 mg (0.50 mmol, 89%) of the title compound as a foam.

¹H-NMR (DMSO-d₆): o: 3.11 (s, 3H, 3′-OCH₃), 3.24 (s, 3H, 1′-OCH₃), 3.64 (dAB, 2H, J=12.3 Hz, J_{5′,6′}=5.9 Hz, H6′), 3.83 (dD, 1H, J=3.9 and 10.3 Hz, H3′), 3.87 (m, 1H, H5′), 4.10 (br, 1H, H4′), 4.48 (dd, 1H, J_{2′,1′}=6.8 Hz, J_{2′,3′}=10.3 Hz, H2′), 4.94 (brs, 1H, 6′-OH), 4.96 (d, J=6.8 Hz, 1H, H1′), 5.03 (brs, 1H, 4′-OH), 7.56 (s, 1H) and 7.75 (s, 1H) (NH₂), 8.72 (s, 1H, H5); ¹³C-NMR (D)MSO-6+D₂O) δ: 160.56 (CO), 157.14 (C3), 146.38 (C5), 98.98 (C1′), 77.68 (C5′), 76.01 (C3′), 63.20 (C4′), 60.83 (C6′ and C2′), 56.33 (3′-OCH₃), 55.15 (1′-OCH₃); ESI-MS pos. HRMS calcd. for C₁₁H₁₉N₄O₆ 303.1305; found 303.1327 [M+H]⁺.

### EXAMPLE 34

### Oligomer Synthesis

As an example for oligomer synthesis, oligos can be assembled on a propanediol containing universal support obviating the need of modified supports (see reference [34]), or can be assembled on commercially available supports. As examples, the new analogues have been used for synthesis of homopolymers, for incorporation within HNA stretches, or for incorporation within stretches of RNA. Hereto, a 0.11 M amidite concentration was used as was done for the HNA building blocks, with a coupling time of 3 minutes. Coupling yields were consistently over 95% and higher. Oligos were purified as was done before (see reference [34]) on a Mono Q ® (Pharmacia) column with a NaCl gradient at pH 12 to disrupt possible secondary structures, but many alternative purification procedures can be used. MS of the isolated oligos were run following gel filtration, RP-HPLC with a 0.025M TEAB containing acetonitrile gradient and occasionally the addition of extra ion exchange beads under TEAH⁺ form to reduce all sodium adducts. Electrospray ionization mass spectrometry (ESI-MS) in negative mode was performed on a quadruple/orthogonal-acceleration time-of-flight (Q/oaTOF) tandem mass spectrometer (qT of 2, Micromass, Manchester, UK) equipped with a standard electrospray ionisation interface. Samples were infused in an acetonitrile:water (1:1) mixture at 3 μL/min. Monoisotopic masses were consistently within 0.5 Da of the calculated masses. Some examples are given below.

TABLE 1
ESMS of some isolated oligomers (for conditions see above).

| | calcd. monoisotopic | |
|---|---|---|
| hexitol sequence^{a} | mass | mass found |
| 1.1 C 1.1 C C 1.1 | 1987.4 | 1987.8 |
| GCG 1.1 A GCG | 2715.6 | 2716.4 |
| (1.1)₁₃ | 4420.9 | 4420.6 |
| G 1.1 G 1.1 ACAC | 2690.6 | 2691.4 |
| 2.2 C 2.2 C C 2.2 | 2029.5 | 2029.8 |
| GCG 2.2 A GCG | 2729.6 | 2729.7 |
| (2.2)₁₃ | 4601.0 | 4601.0 |
| G 2.2 G 2.2 ACAC | 2718.6 | 2718.6 |
| 4.2 C 4.2 C C 4.2 | 2119.5 | 2119.6 |
| GCG 4.2 4.5 GCG | 2819.6 | 2819.7 |
| 4.5 G G 4.5 G 4.5 | 2266.5 | 2266.7 |
| (4.5)₁₃ | 5108.3 | 5108.6 |
| G 1.1 G 1.1 ACAC | 2690.6 | 2691.4 |
| d(TCCTG) (4.5)₆ d(CGCCG) | 5301.1 | 5301.1 |
| (dT 4.2)₆ dT | 4334.9 | 4335.0 |

^{a}All hexitol modifications (1.1, 2.2, 4.2, 4.5) of the here listed sequencesare introduced within 1,5-anhydrohexitol oligomers, with at the 4′-terminal end a 1,3-propanediol phosphate ester moiety (serving as universal terminal), except for the last two sequences where the alkylated hexitol monomers were incorporated within DNA sequences with regular 3′—OH terminal end.

### EXAMPLE 35

### Evaluation of Pyrimidine Hexamers

As an example a study with pyrimidine hexamers was done as depicted in Table 2. When hybridised with complementary HNA, the introduction of the 3′-O-methylated uridine nucleoside (1.1) into a HNA strand results in an increased thermal stability of the duplex compared to the unmodified HNA:HNA duplex (ΔTₘ=+0.6° C./modification), entry A and B, Table 2.

However, this increase is less pronounced than the increase in thermal stability obtained by modifying the nucleobase with a methyl substituent in the 5-position (ΔTₘ=+1.1° C./modification), (compare entry A and D). Hybridisation of the modified ON with complementary ANA results in a duplex with decreased thermal stability compared to the parent ANA:ANA duplex, however, entry C represents fully modified ANA, thus with 6 modifications versus 3 for entry A. Hybridising the modified ONs with complementary RNA corroborates this pattern of thermal stabilisation of the duplexes. The duplex between the 3′-O-methylated ON and complementary RNA is slightly more stable than the corresponding HNA duplex (ΔTₘ=0.2° C./modification), entry A and B, but considerably less stable as compared to the corresponding ANA:RNA duplex (ΔTₘ=−1.1° C./modification, however, this represents fully modified ANA) and the duplex between RNA and the base modified HNA oligo (thymine replacing for uracil) (ΔTₘ=−2.6° C./modification), entry C and D.

TABLE 2
Hybridisation data for hexameric hexitol sequences (6′→4′) with
incorporation of 3 methylated building blocks 1.1 or 2.2.

| | | HNA | ANA | RNA |
|---|---|---|---|---|
| | Sequence | complement | complement | complement |
| A | 1.1 C 1.1 C C 1.1 (HNA) | 52.4 (64)^{a} | 58.8 (71) | 31 (42) |
| B | U C U C C U (HNA) | 50.7 (61.2) | 55 | 30.5 (40) |
| C | U C U C C U (ANA) | 54 | 61.8 (71.2) | 38.4 (47.6) |
| D | T C T C C T (HNA) | 54 | 60.6 | 39 (48) |
| E | 2.2 C 2.2 C C 2.2 (HNA) | 56.7 | 62.7 | 39.9 (49.5) |
| F | 4.5 C 4.5 C C 4.5 (HNA) | 51^{b} | 55.5 | 43 |

^{a}Tₘ's obtained in a buffer consisting of 0.1 M NaCl and 20 mM phosphate, pH 7.4 with a duplex concentration of 4 μM. Numbers in brackets are Tₘ's in a high salt buffer (1.0 M NaCl).
1.1, 2.2 and 4.5 denote the 3′-O-methylated uracil ANA monomer, the 1′-O-methylated thymine HNA monomer and the 1′3′-bis-O-methylated adenine HNA monomer, respectively;
^{b}Tₘ was obtained versus UC containing HNA complement, with 55° C. versus the HNA complement comprising T and C.

For the pyranosylated analogue 2.2 comparison is more straightforward, and the thermal stabilisation is of the same order as for 1.1 when compared to HNA, as well in its pairing with hexitol oligonucleotides (ΔTₘ≈+0.8° C./modification) as with RNA sequences (ΔTₘ≈+0.3° C./modification). Therefore, introduction of 2.2 seems to be slightly more favorable over addition of a HNA monomer, in terms of improved binding. Introduction of a bis-methylated analogue likewise maintained the hybridisation potential versus other hexitol sequences as well as versus RNA sequences.

### EXAMPLE 36

### Evaluation of Octameric Sequences

The results in Table 3 confirm the results of Table 2 (example 35). Thus, incorporation of a 3′-O-methyl-ANA nucleoside (1.1) into a HNA sequence results in a duplex with complementary RNA being thermally more stable than the duplex between the parent HNA and complementary RNA (entry B vs. A). The effect is however less than for the duplexes containing ANA or those containing the 5-methyl modification (entry E with a ΔTₘ/modification=1° C. and C with ΔTₘ/modification=2° C., respectively). However, the modification 2.2 gives a solid increase of 1.6° C. compared with the hexitol T reference (entries C and D) under these circumstances.

TABLE 3
Thermal stability of octameric HNA sequences with a single
incorporation of a methylated analogue 1.1 or 2.2 versus RNA

| Entry | Sequence | Tₘ/° C.^{a} | ΔTₘ/° C.^{b} |
|---|---|---|---|
| A | GCG UA GCG (HNA) | 52 | ref. |
| B | GCG 1.1 A GCG (HNA) | 52.4 | +0.4 |
| C | GCG TA GCG (HNA) | 54 | +2.0 |
| D | GCG 2.2 A GCG (HNA) | 55.6 | +3.6 |
| E | GCG UA GCG (full ANA) | 59.6 | +1.0 |

^{a}Tₘ's towards complementary RNA, obtained in a buffer consisting of 0.1 M NaCl and 20 mM phosphate, pH 7.4 with a duplex concentration of 4 μM;
^{b}ΔTₘ/modification;
1.1 and 2.2 denote the 3′-O-methylated ANA monomer and the 1′-O-methylated HNA monomer, respectively.

### EXAMPLE 37

### Evaluation of Self-Complementary Hexitol Sequences

Further examples are given with self-complementary duplexes of hexitol nucleoside analogues, as shown in Table 4. Incorporation of 3′-O-methyl-ANA nucleosides 1.1 into a self complementary sequence results in considerable stabilisation of the duplex (ΔTₘ=+3° C./modification) exceeding the stabilisation obtained for the substitution of uracil for thymine (ΔTₘ=+2.4° C./modification). This change in stabilisation effect might be an effect of the studied sequence or can be explained by a more continuous run of hydrophobic methyl groups. On the other hand, the effect of the 1′-O-methyl hexitol analogue 2.2 is less impressive in this case, but this can be explained by the high melting temperature of the reference duplex (entry C) which becomes difficult to surmount. However, the analogue still improves the hybridisation properties.

TABLE 4
Thermal stability of self complementary HNA sequences containing
3′- or 1′-O-methyl modifications (1.1 or 2.2).

| Sequence | Tₘ/° C. | ΔTₘ/° C.^{a} |
|---|---|---|
| G U G U A C A C | 65.0 | ref. |
| G 1.1 G 1.1 A C A C | 76.7 | +3 |
| G T G T A C A C | 74.5 | +2.4/ref. |
| G 2.2 G 2.2 A C A C | 76.9 | +0.6 |

Tₘ's obtained in 0.1 M NaCl, 20 mM phosphate, pH 7.4 with an oligo concentration of 8 μM (4 μM of duplex).
^{a}ΔTₘ/modification.

Generally, the ONs containing the 3′-O-methyl derivative (1.1) show a small increase in thermal stability towards complementary sequences as compared to HNA, except in the case of a self-complementary sequence for which an increase in thermal stability of 3° C. per modification is observed. Compared to altritol nucleic acids (ANA), however, the 3′-O-methylation causes a decrease in thermal stability of duplexes between a modified ON and a complementary target, especially when targeting RNA. The introduction of a hydrophobic moiety at the rim of the minor groove does not seem to have a large destabilising effect and the slightly decreased affinity of the methylated analogue as compared to parent ANA towards complementary sequences is probably due to the reduced ability to form hydrogen bonds, i.e. lost ability to act as a proton donor. These examples suggest that it is possible to derivate the 3′-hydroxyl group in ANA without significantly affecting the thermal stability of the duplexes with complementary sequences leaving room for alkylation using different alkyl moieties. In addition, the examples with the 1′-O-methyl nucleoside analogues 2.2 indicate the improved hybridisation potential with higher affinity for RNA in comparison with well-known HNA, while at the same time having technically more favorable monomers.

### EXAMPLE 38

### Incorporation within RNA Oligomers

Previous studies have indicated the hexitol analogues to resemble more the ribonucleosides (see references [2], [3], [4] and [18]). As the pre-organized structure of a hexitol nucleoside analogue can possibly have effect on the remaining part of the sequence, incorporation of a single modification of either 1.1 or 2.2 or 4.2 or for comparative purposes of the 1,5-anhydrohexitol nucleoside analogues of uridine and thymidine (hU or hT) into natural RNA monomers is studied within different sequence context, and the

TABLE 5
Thermal stability of RNA sequences (5′→3′)
containing a single modified building block hybridized
to complementary RNA nonamers.

| Entry | Sequence | Tₘ/° C.^{a} | ΔTₘ/° C.^{b} |
|---|---|---|---|
| A | GCG U U U GCG | 51.4 (59.3) | reference |
| B | GCG U U_{OMe} U GCG | 53.0 (60.8) | +1.6 (1.5) |
| C | GCG U hU U GCG | 54.4 (61.6) | +3.0 (2.3) |
| D | GCG U 1.1 U GCG | 55.4 (62.4) | +4.0 (3.1) |
| E | GCG U hT U GCG | 55.8 | +4.4 |
| F | GCG U 2.2 U GCG | 54.0 | +2.6 |
| G | GCG U 4.2 U GCG | 52.3 | +0.9 |
| H | GCU G U G UCG | 55.9 (62.8) | reference |
| I | GCU G U_{OMe} G UCG | 57.3 (64.6) | +1.4 (1.8) |
| J | GCU G hU G UCG | 57.1 (64.7) | +1.2 (1.9) |
| K | GCU G 1.1 G UCG | 59.3 (66.5) | +3.4 (3.7) |
| L | GCU G hT G UCG | 58.2 | +2.3 |
| M | GCU G 2.2 G UCG | 56.3 | +0.4 |
| N | GCU G 4.2 G UCG | 55.9 | +0 |
| O | GCA C U C ACG | 56.9 (63.8) | reference |
| P | GCA C U_{OMe} C ACG | 58.0 (65.1) | +1.1 (1.3) |
| Q | GCA C hU C ACG | 60.0 (66.9) | +3.1 (3.1) |
| R | GCA C 1.1 C ACG | 60.8 (67.7) | +3.9 (3.9) |
| S | GCC A U A CCG | 57.1 (64.4) | reference |
| T | GCC A U_{OMe} A CCG | 58.8 (66.4) | +1.7 (2.0) |
| U | GCC A hU A CCG | 57.2 (64.9) | +0.1 (0.5) |
| V | GCC A 1.1 A CCG | 58.9 (66.2) | +1.8 (1.8) |

^{a}Tₘ's towards complementary RNA, obtained in a buffer consisting of 0.1 M NaCl (respectively 1 M NaCl) and 20 mM phosphate, pH 7.4 with a duplex concentration of 4 μM;
^{b}ΔTₘ/modification;
U_{OMe} characterizes 2′-O-methyluridine and hU and hT characterize a 1,5-anhydrohexitol monomer.

obtained modified oligos are evaluated versus RNA complementary sequences (table 5). It is documented that a single modified nucleotide when incorporated into natural nucleic acids may induce local geometry changes over several neighbouring basepairs. Therefore, in our example the modified nucleotides are incorporated within as well UpXpU, CpXpC, ApXpA as GpXpG motifs. Nevertheless, strong hybridizing complexes are obtained, indicative of a preorganized structure fitting the dsRNA A-type duplex. The thermal stabilities are compared with incorporation of 2′-O-methylated uridine monomers at the same position. As expected from the literature, the 2′-O-methyluridine containing oligos (see references [21] and [22]), display increased affinity for the RNA complement over the non-methylated reference oligos. However, likewise a systematic increase in affinity is noticed of both the methylated and non-methylated hexitol modification containing oligos for their respective complementary sequences. The oligos with the plain hexitol modification (hU) surpass the reference RNA oligos in affinity for their target, while the oligos containing the 3′-O-methylated altrohexitol modification 1.1 prove to be endowed with an even better affinity, surpassing the Tm values for the reference oligos mostly by 3 to 4° C. except for the ApXpA motif (entry V, +1.8° C.). However, for this motif incorporation of hexitol U itself did not show any influence on Tm (entry U). The beneficial effects of analogues like 2.2, and the maintained hybridisation potential of analogues like 4.2 can be further deduced from table 5.

As pointed out before, we have to keep the change in conformation in mind, which takes place by incorporation of the modification. Therefore, multiple incorporation of these modified building blocks can result in even higher stabilities of the formed complexes and thus higher affinities of the backbone modified oligos for RNA. Clearly, the examples have shown it is possible to incorporate the new modified monomers into RNA oligonucleotides, without comprising the affinity for their respective RNA target.

Taking together, the present invention eliminates the problem of the supplementary protecting group as necessary in altritol nucleic acids (ANA) by alkylation of the [S]-hydroxyl group which is liberated upon opening of the allitol epoxide by introduction of the heterocyclic base moiety (see reference [14]). Such alkylation reaction paves the way for a series of new nucleoside analogues, for example the 3′-O-methyl altritol nucleoside analogues (1.Y), or more generally 3′-O-alkyl altritol nucleoside analogues as further exemplified by the analogues 14.Y and 15.Y), all useful for incorporation into oligonucleotides (Scheme III).

In addition, the present invention details the synthesis of 3′-deoxy-1′-O-methyl hexitol nucleoside analogues (2.Y), preferably using ubiquitous methylglucoside as starting material, eliminating the need for reductive deoxygenation of the Cl-position.

In addition, the 3′-hydroxy-1′-O-methyl hexitol nucleoside analogues 3.Y, as well as the 1′,3′-bis-O-methyl hexitol nucleoside analogues 4.Y, and the 3‘-O-alkyl-l’-O-methyl hexitol nucleoside analogues have been invented and are prepared likewise preferably starting from methylglucoside. All new alkylated nucleoside analogues can be functionalized to allow incorporation into oligonucleotides, and the newly constructed oligomers all maintain or improve the pairing potential for RNA oligonucleotides.

### REFERENCES

[1] Van Aerschot, A.; Verheggen, I.; Hendrix, C.; Herdewijn, P. Angew. Chem. 1995, 107, 1483-1485. Angew. Chem. Int. Ed. 1995, 34, 1338-1339.

[2] Hendrix, C.; Rosemeyer, H.; Verheggen, I.; Seela, F.; Van Aerschot, A.; Herdewijn, P. Chemisty: European J., 1997, 3, 110-120.

[3] Hendrix, C.; Rosemeyer, H.; De Bouvere, B.; Van Aerschot, A.; Seela, F.; Herdewijn, P. Chemistry: European J., 1997, 3, 1513-1520.

[4] Boudou, V.; Kerremans, L.; De Bouvere, B.; Schepers, G.; Busson, R.; Van Aerschot, A.; Herdewijn, P. Nucleic Acids Res., 1999, 27, 1450-1456.

[5] Martin, P. Helv. Chim. Acta 1995, 78, 486-504.

[6] Tarköy, M.; Bolli, M.; Schweizer, B.; Leumann, C. Helv. Chim. Acta 1993, 76, 481-510.

[7] Singh, S. K.; Nielsen, P.; Koshlkin, A. A.; Wengel, J. Chem. Commun. 1998, 455-456.

[8] Herdewijn, P. Liebigs Ann. 1996, 1337-1348.

[9] Vandermeeren, M.; Preveral, S.; Janssens, S.; Geysen, J.; Saison-Behmoaras, E.; Van Aerschot, A.; Herdewijn, P. Biochem Pharmacol., 2000, 59, 655-663.

[10] Flores, Maria V. C.; Atkins, D.; Stewar, T. S.; Van Aerschot, A.; Herdewijn, P. Parasitol. Res. Series, 1999, 85, 864-866.

[11] De Winter, H.; Lescrinier, E.; Van Aerschot, A.; Herdewijn, P. J. Am. Chem. Soc. 1998, 120, 5381-8394.

[12] Allart, B.; Khan, K.; Rosemeyer, H.; Schepers, G.; Hendrix, C.; Rothenbacher, K.; Seela, F.; Van Aerschot, A.; Herdewijn, P. Chemistry: European J., 1999, 5, 2424-2431.

[13] Hossain, N.; Wroblowsld, B.; Van Aerschot, A.; Rozenski, J.; De Bruyn, A.; Herdewijn, P. J. Org. Chem. 1998, 63, 1574-1582.

[14] Allart, B.; Busson, R.; Rozenski, J.; Van Aerschot, A.; Herdewijn, P. Tetrahedron, 1999, 55, 6527-6546.

[15] Verheggen, I.; Van Aerschot, A; Toppet, S.; Snoeck, R.; Janssen, G.; Claes, P.; Balzarini, J.; De Clercq, B.; Herdewijn, P. J. Med. Chem., 1993, 36, 2033-2040.

[16] Andersen, M. W.; Daluge, S. M.; Kerremans, L.; Herdewijn, P. Tetrahedron Lett. 1996, 37, 8147-8150.

[17] De Bouvere, B.; Kerremans, L.; Rozensid, J.; Janssen, G.; Van Aerschot, A.; Claes, P.; Busson, R.; Herdewijn, P. Liebigs Ann./Receuil, 1997, 1453-1461.

[18] Herdewijn, P. Biochem. Biophys. Acta 1999, 1489, 167-179.

[19] Uhlman, E.; Peyman, A. Chem. Rev. 1990, 90, 543-584.

[20] Carbohydrate Modifications in Antisense Research, ACS Symposium Series 580 (Eds.: Sanghvi, Y. S.; Cook, P. D.), Washington, 1994.

[21] Inoue, H.; Hayase, Y.; Imma, A.; Iwai, S.; Miura, K and Ohtsuka, E., Nucleic Acids Res. 1987, 15, 6131-6148.

[22] Sproat, B.; Lamond, A.; Beijer, B.; Neuner, P.; Ryder, U. Nucleic Acids Res., 1989, 17, 3373-3386.

[23] P. Martin, Helv. Chim. Acta-1995, 78, 486-504.

[24] Griffey, R. H. et al., J. Med. Chem. 1996, 39, 5100-5109.

[25] Prakash, T. P.; Manoharan, M.; Kawasali, A. M.; Lesnik, E. A; Owens, S. R. and Vasquez, G. et al., Org. Lett., 2000, 2, 3995-3998.

[26] Pfundheller, H. M.; Koshkin, A. A.; Olsen, C. E.; Wengel, J. Nucleosides Nucleotides, 1999, 18, 2017-2030.

[27] Wang, G.; Seifert, W. E. Tetrahedron Lett., 1996, 37, 6515-6518.

[28] Lin, T-S.; Gao, Y-S.; Mancini, W. R. J. Med. Chem., 1983, 26, 1691-1696.

[29] Luyten, I. and Herdewijn, P. Eur. J. Med Chem., 1998, 33, 515-578.

[30] Herdewijn, P. Antisense & Nucleic Acid Drug Dev., 2000, 10, 297-310.

[31] Loakes, D. Nucleic Acids Res., 2001, 29, 2437-2447.

[32] Richtmeyer, N. K; Hudson, C. S. J. Am. Chem. Soc., 1941, 63, 1727-1731.

[33] Rosenfeld, D. A.; Richtmeyer, N. K; Hudson, C. S. J. Am. Chem. Soc., 1948, 70, 2201-2206.

[34] Van Aerschot, A.; Saison-Behmoaras, E.; Rozenski, I.; Hendrix, C.; Schepers, G.; Verhoeven, G.; Herdewijn, P. Bull. Soc. Chim. Belges, 1995, 104, 717-720.

[35] Dagle, J. M.; Walder, J. A.; Weeks, D. L. Nucleic Acids Res. 1990, 18, 4751-4757.

[36] Dagle, J. M.; Weeks, D. L.; Walder, J. A. Antisense Res. and Dev. 1991, 1, 11-20

[37] Eder, P. S.; Walder, J. A. J. Biol. Chem. 1991, 266, 6472-6479.

[38] Dagle, J. M.; Andracki, M. E.; DeVine, R. I.; Walder, J. A. Nucleic Acids Res. 1991, 19, 1805-1810.

[39] Matteucci, M. D. and Caruthers, M. H., J. Am. Chem. Soc. 1981, 103, 3185-3191.

[40] Froehler, B.; Ng, P.; Matteucci, M. D., Nucleic Acids Res. 1986, 14, 5399-5407.

[41] Methods in Molecular Biology, vol. 20, Protocols for oligonucleotides and analogues, S. Agrawal ed., Humana Press, Totowa, N.J., U.S.A.

[42] Ti, G. S.; Gaffney, B. L.; Jones, R. A., J. Am. Chem. Soc. 1982, 104, 1316-1319.

We claim:

1. An alkylated 1,5-anhydro-2-deoxy-altro-hexitol nucleoside compound, represented by the general formula I 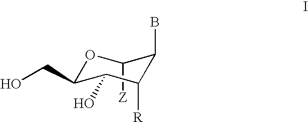
wherein:
B is a heterocyclic ring derived from the group consisting of pyrimidine and purine bases, or from the group of nitroazole and azole carboxamide bases;
Z is hydrogen or O-methyl; and
R is an O-alkyl group, with:
alkyl being a straight or branched chain, saturated or unsaturated, substituted or unsubstituted hydrocarbon radical having from 1 to 6 carbon atoms;
or R has the formula: 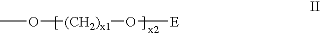
x1 is from 2 to 6;
x2 is independently from 0 to 6;
E is C₁-C₆ alkyl or N(Q₁)(Q₂);
each Q₁ and Q₂ is, independently, hydrogen, C₁-C₆ alkyl, substituted alkyl, a nitrogen protecting group, a tethered or untethered conjugate group; or Q₁ and Q₂, together, are joined in a nitrogen protecting group;
or E is hydrogen, provided x2 is different from zero;
or R is hydrogen, provided Z is O-methyl;
or R is hydroxyl, provided B is a heterocyclic ring derived from the group of nitroazole and azole carboxamide bases.

2. A compound according to claim 1 wherein
R is an O-alkyl group, with:
alkyl being a straight or branched chain, saturated or unsaturated, substituted or unsubstituted hydrocarbon radical having from 1 to 6 carbon atoms;
or R has the formula: 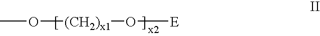
x1 is from 2 to 6;
x2 is independently from 0 to 6;
E is C₁-C₆ alkyl or N(Q₁)(Q₂);
each Q₁ and Q₂ is, independently, hydrogen, C₁-C₆ alky, substituted alkyl, a nitrogen protecting group, a tethered or untethered conjugate group; or Q₁ and Q₂, together, are joined in a nitrogen protecting group;
or E is hydrogen, provided x2 is different from zero.

3. A compound according to claim 1 wherein R is O-methyl.

4. A compound according to claim 1 wherein R is —O—CH₂—CH₂—O—CH₃.

5. A compound according to claim 1 wherein R is —O—CH₂—CH₂—CH₂—NH₂.

6. A compound as claimed in in claim 1 wherein R is —O—CH₂—CH₂—O—N(CH₃)₂.

7. A compound as claimed in claim 1 wherein Z is hydrogen and R is O-methyl.

8. A compound as claimed in claim 1 wherein Z is hydrogen and R is —O—CH₂—CH₂—O—CH₃.

9. A compound as claimed in claim 1 wherein Z is hydrogen and R is —O—CH₂—CH₂—CH₂—NH₂.

10. A compound as claimed in claim 1 wherein Z is hydrogen and R is —O—H₂—CH₂—O—N(CH₃)₂.

11. A compound as claimed in claim 1 wherein Z is O-methyl and R is O-methyl.

12. A compound as claimed in claim 1 wherein Z is O-methyl and R is —O—CH₂—CH₂—O—CH₃.

13. A compound as claimed in claim 1 wherein Z is O-methyl and R is —O—CH₂—CH₂—CH₂—NH₂.

14. A compound as claimed in claim 1 wherein Z is O-methyl and R is —O—CH₂—CH₂—O—N(CH₃)₂.

15. A compound as claimed in any one of the claims 1 through 14 wherein B is a heterocyclic ring derived from the group consisting of pyrimidine and purine bases.

16. A compound as claimed in any one of the claims 1 through 14 wherein B is a heterocyclic ring derived from the group consisting of nitroazole and azole carboxamide bases.

17. An oligomer comprising or containing in part at least one of the hexitol nucleoside analogues as claimed in any one of the claims 1 through 16.

18. An oligomer comprising hexitol nucleoside analogues as claimed in any one of the claims 1 through 16, and deoxyribose or ribose nucleotides having the general formula III. 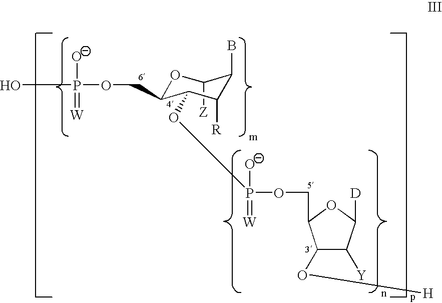
wherein:
m, n and p are integers;
p≧2;
each m≧1, provided that mₗ may be zero;
each n≧1, provided that nₚ may be zero;
each B independently is a heterocyclic ring derived from the group consisting of pyrimidine and purine bases, or from the group of nitroazole and azole carboxamide bases;
each D independently is a heterocyclic ring which is derived from a pyrimidine or purine base;
each Y independently is hydrogen or hydroxyl;
each Z independently is hydrogen or O-methyl;
each R independently is an O-alkyl group, with:
alkyl being a straight or branched chain, saturated or unsaturated, substituted or unsubstituted hydrocarbon radical having from 1 to 6 carbon atoms;
or each R independently has the formula: 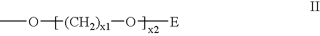
x1 is from 2 to 6;
x2 is independently from 0 to 6;
E is C₁-C₆ alkyl or N(Q₁)(Q₂);
each Q₁ and Q₂ is, independently, hydrogen, C₁-C₆ alkyl, substituted alkyl, a nitrogen protecting group, a tethered or untethered conjugate group; or Q₁ and Q₂, together, are joined in a nitrogen protecting group;
or E is hydrogen, provided x2 is different from zero;
or each R is hydrogen, provided Z is O-methyl;
or each R is hydroxyl, provided B is a heterocyclic ring derived from the group of nitroazole and azole carboxamide bases;
each W independently represents oxygen or sulfur; the 5′-end, respectively the 6′-end, of the oligonucleotide may optionally be dephosphorylated and the 3′-end, respectively the 4′-end, may optionally be phosphorylated;
and all possible salt forms thereof.

19. An oligomer comprising hexitol nucleoside analogues as claimed in any one of the claims 1 through 16, having the general formula IV 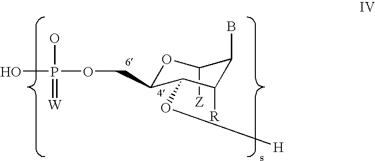
wherein:
s is an integer with s≧2;
each B independently is a heterocyclic ring derived from the group consisting of pyrimidine and purine bases, or from the group of nitroazole and azole carboxamide bases;
each Z independently is hydrogen or O-methyl;
each R independently is an O-alkyl group, with:
alkyl being a straight or branched chain, saturated or unsaturated, substituted or unsubstituted hydrocarbon radical having from 1 to 6 carbon atoms;
or each R independently has the formula: 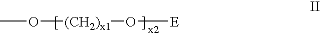
x1 is from 2 to 6;
x2 is independently from 0 to 6;
E is C₁-C₆ allyl or N(Q₁)(Q₂);
each Q₁ and Q₂ is, independently, hydrogen, C₁-C₆ alkyl, substituted alkyl a nitrogen protecting group, a tethered or untethered conjugate group; or Q₁ and Q₂, together, are joined in a nitrogen protecting group;
or E is hydrogen, provided x2 is different from zero;
or each R is hydrogen, provided Z is O-methyl;
or each R is hydroxyl, provided B is a heterocyclic ring derived from the group of nitroazole and azole carboxamide bases;
each W independently represents oxygen or sulfur;
the 6′-end, of the oligonucleotide may optionally be dephosphorylated and the 4′-end, may optionally be phosphorylated;
and all possible salt forms thereof.

20. A complex comprising a first oligomer as claimed in any one of claims 17 to 19 and either a complementary single-stranded or double-stranded natural oligonucleotide or a self-complementary second oligomer as claimed in any of the claims 17 to 19, wherein each of the studs in the complex may be of the same or of a different length.

21. An oligomer as claimed in any one of claims 17 to 19 or a complex according to claim 20 for use as a medicine.

22. A pharmaceutical composition comprising as an active ingredient a therapeutically effective amount of an oligomer as claimed in any one of claims 17 to 19 or a complex according to claim 20, and a pharmaceutically acceptable carrier.

23. A process of preparing a pharmaceutical composition as claimed in claim 22, characterized in that a therapeutically effective amount of an oligomer as claimed in any one of claims 17 to 19 or a complex as claimed in claim 20, is mixed with a pharmaceutically acceptable carrier.

24. The use of one or more of the nucleoside analogues as claimed in any one of claims 1 through 16 for incorporation into oligonucleotides.

25. The use of an oligomer as claimed in any one of claims 11 to 19 or of a complex as claimed in claim 20, in molecular biology and/or genetic engineering.

26. Uses according to claim 25 comprising hybridisation, isolation of nucleic acids, isolation of DNA or RNA fragments, site-specific DNA modification, mapping.

27. The use of an oligomer as claimed in any one of claims 17 to 19 or of a complex as claimed in claim 20, in antisense strategies, which comprise the steps of hybridisation, isolation of nucleic acids, site-specific DNA modification, and therapeutics.

28. The use of an oligomer as claimed in claim 27 further comprising determining the diagnosis of a disease.

29. The use of an oligomer as claimed in claim 27 for use as a medicine.

30. An antisense compound, said antisense compound being an oligomer as claimed in any one of claims 17 to 19 or of a complex as claimed in claim 20.
